(19) United States
(12) Patent Application Publication
(10) Pub. No.: US 2021/0093245 A1
(43) Pub. Date: Apr. 1, 2021

(54) SYSTEM AND METHOD FOR COLLECTING BIOLOGICAL DATA

(71) Applicant: BIOS HEALTH LTD, Cambridgeshire (GB)

(72) Inventors: Oliver ARMITAGE, Cambridge Cambridgeshire (GB); Emil HEWAGE, Aberdeen Aberdeenshire (GB); Joned SAWAR, Cambridge Cambridgeshire (GB); Sudhakaran SHUNMUGAM, Cambridge Cambridgeshire (GB); Tristan EDWARDS, Cambridge Cambridgeshire (GB); Kalon HEWAGE, St-Leonards-On-Sea Sussex (GB)

(21) Appl. No.: 17/042,057

(22) PCT Filed: Mar. 27, 2019
(86) PCT No.: PCT/GB2019/050872

§ 371 Date: Sep. 25, 2020

(30) Foreign Application Priority Data
Mar. 27, 2018 (GB) 1804955.1
Jul. 25, 2018 (GB) 1812137.6

(51) Int. Cl. A61B 5/24; A61B 5/00

(52) U.S. Cl. CPC A61B 5/24; A61B 5/4058; A61B 5/6865; A61B 2560/0214; A61B 5/486; A61B 5/0004; A61B 5/0024

(57) ABSTRACT

A system for obtaining biological data from a subject animal, the system comprising at least one neural transducer embedded internally within the subject animal and arranged for interaction with nerves of the subject animal, at least one sensor arranged for sensing data of a parameter of the subject animal, and an external module mounted externally on the subject animal and connected by a wired connection to at least one of the at least one neural transducer or the at least one sensor. The wired connection comprises a sealed and ported through skin interface device.

The present application relates to a system and method for collecting biological data, and in particular for collecting biological data from a living subject.

### BACKGROUND

In the fields of biological and medical research it is often required to collect biological data from a living animal subject in vivo using a sensor embedded within the body of the subject. There are many different types of biological data which may be collected in this way. One example of such biological data is neural data. Neural data comprises measurements of activity in the nerves of the subject, which measurements are generally gathered using electrodes implanted in the body of the subject on, or close to, the nerves of interest by extra-or intra- cellular recording .

A known approach to collecting biological data from a living animal subject is to use one or more small sensors which are wholly implanted within the body of the subject and each comprise a wireless transceiver and a power source. In operation the implanted sensors uses their wireless transceivers to communicate the collected biological data to a separate external data recording system for subsequent analysis.

Another known approach is to physically connect one or more implanted sensors to an external data recording system through a tether comprising electrical conductors. In operation power is provided to the implanted sensors through the tether, and the sensors communicate the collected biological data through the tether to the external data recording system for subsequent analysis.

However, there are problems with these approaches. When wholly implanted sensors are used, the working lifetime of each sensor is limited to the length of time for which the power source of that sensor, typically a battery, can provide enough power to operate the sensor. This can require an undesirable trade-off having to be made regarding the operation of the wireless transceiver, where it may be difficult to transmit the collected biological data so that it can be reliably received by the external data recording system without using a transmission power level which is so high that the working lifetime of the sensor is undesirably shortened. The problem of limited sensor lifetime is a particular problem in the collection of neural data because neural data generally requires a very large volume of data to be collected at a high level of fidelity, so that wireless transmission of the neural data tends to consume a large amount of power. Further, the low voltage levels and high data rates of the sensed nerve activity generally require high performance sensing and processing circuitry with relatively high power demands. As a result, neural data acquisition systems generally use the tethered approach, or collect neural data only for short periods.

When a tether is used the restraint placed on the movements and activity of the subject by the tether may prevent natural behavior of the subject, or awareness of the tether may cause the subject to change its behavior, so that the collected data is not representative of the usual behavior of the subject in their natural environment. In some cases this problem may be made worse by requirements to further restrain or immobilize the subject in order to prevent damage to or interference with the tether, and/or injury to the subject. Further, the need to pass electrical conductors through the subjects skin or to have a permanently exposed section of soft tissue in order to provide power and data connections between the implanted sensors and the tether generates an infection risk at the skin penetration site or sites, and any resulting infections can place the subject at risk, and also can effect both the natural behavior of the subject and the biological processes being measured, reducing the usefulness of the collected data.

As a result of these problems neural data in particular gathered by known techniques is either of short duration or of a subject with a limited movement range. Long term recordings of neural data for freely moving subjects of more than a few hours duration are not available.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter; variants and alternative features which facilitate the working of the invention and/or serve to achieve a substantially similar technical effect should be considered as falling into the scope of the invention disclosed herein.

In a first aspect, the present disclosure provides a system for obtaining biological data from a subject animal, the system comprising: at least one neural transducer embedded internally within the subject animal and arranged for interaction with nerves of the subject animal; at least one sensor arranged for sensing data of a parameter of the subject animal; and an external module mounted externally on the subject animal and connected through a port to at least one of the at least one neural transducer or the at least one sensor; wherein the through port connection comprises a sealed and ported through skin interface device.

Preferably, the through port connection is a wired connection, and preferably an electrical wired connection.

Preferably, the through port connection is a fluid or gas passage channel.

Preferably, the through port connection is an optical fiber.

Preferably, the neural transducer is a neural sensor arranged for sensing neural data from nerves of the subject animal.

Preferably, the system further comprises: a wireless communication unit for wirelessly sending the sensed neural data to a data store; means for sending the sensed parameter data to the data store; and a power supply; wherein the external module comprises at least one of the wireless communication unit and the power supply.

Preferably, the data store is arranged to store the sensed neural data together with associated time data, and is arranged to store the sensed parameter data together with associated time data.

Preferably, the external module comprises the power supply and the wireless communication unit.

Preferably, the external module comprises at least one of: a data processor arranged for processing the neural data; a data storage arranged for storing the neural data.

Preferably, the system further comprises at least one wireless communication device for wirelessly receiving the sensed neural data from the wireless communication unit.

Preferably, the system further comprises at least one local computer arranged to record the sensed neural data received by the at least one wireless communication device and the sensed parameter data, and to send the sensed neural data and the sensed parameter data to the data store together with associated time data.

Preferably, the at least one local computer is arranged to calculate metadata based on the sensed neural data and associated time data, and the sensed parameter data and associated time data, and to send the metadata to the data store.

Preferably, the system further comprises the data store.

Preferably, the at least one sensor comprises at least one sensor embedded internally within the subject animal.

Preferably, the at least one sensor embedded internally within the subject animal comprises at least one of: a glucose sensor; a heart rate sensor, an ElectroCardioGram (ECG) sensor, a blood pressure sensor, a vascular pressure sensor, an airway pressure sensor; an intrapleural pressure sensor; a gastric activity sensor; a gastric PH sensor, an Electro Muscular Graph (EMG) sensor.

Preferably, the at least one sensor comprises at least one sensor mounted externally on the subject animal.

Preferably, the at least one sensor mounted externally on the subject animal comprises a motion sensor.

Preferably, the external module comprises a data processor arranged for generating the time data associated with the sensed neural data.

Preferably, the system comprises at least one active device arranged for operating to produce a change in the subject animal.

Preferably, wherein the at least one active device comprises the neural transducer.

Preferably, the at least one active device is arranged to operate in response to an instruction.

Preferably, the instruction is based on the sensed parameter data.

Preferably, the instruction is time based.

Preferably, the instruction is generated, based on the sensed parameter data, by the at least one sensor.

Preferably, the system comprises the data store, and the instruction is generated, based on the sensed parameter data, by the data store.

Preferably, the instruction is received by the system from another system or a user.

Preferably, the system further comprises at least one active device arranged for operating to produce a change in the subject animal, arranged to operate in response to an instruction which is generated by the local computer based on the sensed parameter data.

Preferably, the at least one active device is arranged to operate autonomously.

Preferably, the at least one active device is arranged to send activity data to a data store; and the data store is arranged to store the activity data in association with associated time data.

Preferably, the at least one active device comprises a neural stimulator.

Preferably, the at least one active device comprises a substance delivery device arranged to administer at least one dose of at least one substance to the subject animal.

Preferably, the substance delivery device is arranged to administer at least one of: pharmaceuticals; gene therapies.

Preferably, the substance delivery device is arranged to administer a viral vector treatment.

Preferably, the at least one active device further comprises a neural stimulator; and the viral vector treatment is arranged to enable hypersensitivity or hyposensitivity to neurostimulation in at least some areas of the body of the subject animal.

Preferably, the system comprises a static part not mounted on the subject animal.

Preferably, the static part comprises at least one environmental modification device arranged for operating to produce a change in the environment experienced by the subject animal.

Preferably, the at least one environmental modification device is arranged to operate in response to an instruction.

Preferably, the instruction is based on the sensed parameter data.

Preferably, the instruction is time based.

Preferably, the instruction is generated, based on the sensed parameter data, by the at least one sensor.

Preferably, the system comprises the data store, and the instruction is generated, based on the sensed parameter data, by the data store.

Preferably, the instruction is received by the system from another system or a user.

Preferably, the system comprises a static part not mounted on the subject animal and comprising at least one environmental modification device arranged for operating to produce a change in the environment experienced by the subject animal, arranged to operate in response to an instruction which is generated by the local computer based on the sensed parameter data.

Preferably, the at least one environmental modification device is arranged to operate autonomously.

Preferably, the at least one environmental modification device is arranged to send activity data to a data store; and the data store is arranged to store the activity data in association with the associated time data.

Preferably, the at least one environmental modification device is arranged to provide food to the subject animal.

Preferably, the at least one sensor is part of the static part.

Preferably, the at least one sensor comprises at least one video camera.

Preferably, the data store is part of the static part

Preferably, the system comprises a static part not mounted on the subject animal and the at least one wireless communication device is part of the static part.

Preferably, the system comprises a static part not mounted on the subject animal and the at least one local computer is part of the static part.

Preferably, the data store is arranged to store the meta data and associated time data together with the sensed neural and associated time data, and sensed parameter data and associated time data.

Preferably, the data store is arranged to enable stored neural data and parameter data to be referenced across multiple sensors or, based on time period or based on stored event data.

Preferably, the data store is arranged to store data relating to multiple subject animals and to enable stored neural data and parameter data to be referenced by subject animal.

Preferably, the system is arranged to generate and send notifications in response to the identification of predetermined events in the sensed parameter data and/or the sensed neural data.

Preferably, the system is arranged to generate and send notifications in response to the identification of predetermined events in the sent activity data.

Preferably, the notifications are push notifications.

Preferably, the system further comprises a user front end arranged to enable viewing of the data stored in the data store.

Preferably, the user front end is arranged to enable the neural data and parameter data to be viewed together in a time synchronous manner.

Preferably, the user front end is arranged to enable the neural data and parameter data to be viewed in a time synchronous manner together with data regarding other events.

Preferably, the other events comprise at least one of: delivered neural stimulation; delivered treatments; other events.

Preferably, the neural transducer is a neural sensor arranged for sensing neural data from nerves of the subject animal, and the system further comprises: at least one machine learning model arranged to receive neural data and to determine at least one physiological parameter value or bodily variable based upon the neural data.

Preferably, the machine learning model is arranged to determine at least one bodily variable based upon the neural data, wherein the at least one bodily variable is not directly, or not easily, measurable.

Preferably, the machine learning model is arranged to determine at least one bodily variable based upon the neural data, wherein the at least one bodily variable is not directly, or not easily, measurable by observation of the subject animal.

Preferably, the system further comprises a display means arranged to display data relating to an intermediate state of the machine learning data together with at least one of the neural data and the determined at least one physiological parameter value or bodily variable.

Preferably, the display means is arranged to display data relating to an intermediate state of the machine learning data together with both the neural data and the determined at least one physiological parameter value or bodily variable.

Preferably, the system is further arranged to calculate the at least one physiological parameter value or bodily variable based upon the data sensed by the at least one sensor; and to also display data the calculated at least one physiological parameter value or bodily variable.

Preferably, the different displayed data are synchronous.

Preferably, the display means is arranged to display data relating to an intermediate state of the machine learning data as a reduced dimensionality plot.

Preferably, the reduced dimensionality plot is at least one of: a t-SNE plot; a PCA plot; an ICA plot; or an Isomap.

Preferably, the display means is arranged to display data relating to an intermediate state of the machine learning data representing equivalence to neural populations, neural events, or neural or non-neural biomarkers, or combinations of neural and non-neural biomarkers.

Preferably, the display means is arranged to display data relating to an intermediate state of the machine learning data as vectors of classes.

Preferably, the at least one physiological parameter value or bodily variable is at least one of: heartrate of the subject animal; activity of the subject animal; temperature of the subject animal; blood glucose level of the subject animal; any vital sign of the subject animal; any physiological measurement of the whole of the subject animal, a body part of the subject animal or a sub-part of the subject animal; any data representative of a state of the whole of the subject animal, a body part of the subject animal, or a sub-part of the subject animal.

In a second aspect, the present disclosure provides a system for obtaining biological data from a subject animal, the system comprising: at least one neural transducer embedded internally within the subject animal and arranged for interaction with nerves of the subject animal; at least one active device arranged for producing a change in the subject animal; and an external module mounted externally on the subject animal and connected through a port to at least one of the at least one neural transducer or the at least one active device; wherein the through port connection comprises a sealed and ported through skin interface device.

Preferably, the through port connection is a wired connection, and preferably an electrical wired connection.

Preferably, the through port connection is a fluid or gas passage channel.

Preferably, the through port connection is an optical fiber.

Preferably, the neural transducer is a neural sensor arranged for sensing neural data from nerves of the subject animal.

Preferably, the system further comprises: a wireless communication unit for wirelessly sending the sensed neural data to a data store; and a power supply; wherein the external module comprises at least one of the wireless communication unit and the power supply.

Preferably, the data store is arranged to store the sensed neural data together with associated time data.

Preferably, wherein the external module comprises the power supply and the wireless communication unit.

Preferably, the external module comprises at least one of: a data processor arranged for processing the neural data; a data storage arranged for storing the neural data.

Preferably, the system further comprises at least one wireless communication device for wirelessly receiving the sensed neural data from the wireless communication unit.

Preferably, the system further comprises at least one local computer arranged to record the sensed neural data received by the at least one wireless communication device, and to send the sensed neural data to the data store together with associated time data.

Preferably, the at least one local computer is arranged to calculate metadata based on the sensed neural data and associated time data, and to send the metadata to the data store.

Preferably, the system further comprises the data store.

Preferably, the system further comprises at least at least one sensor embedded internally within the subject animal and arranged for sensing data of a parameter of the subject animal and sending the sensed parameter data to the data store.

Preferably, the at least one sensor embedded internally within the subject animal comprises at least one of: a glucose sensor; a heart rate sensor, an ElectroCardioGram (ECG) sensor, a blood pressure sensor, a vascular pressure sensor, an airway pressure sensor; an intrapleural pressure sensor; a gastric activity sensor; a gastric PH sensor, an Electro Muscular Graph (EMG) sensor.

Preferably, the at least one sensor comprises at least one sensor mounted externally on the subject animal and arranged for sensing data of a parameter of the subject animal and sending the sensed parameter data to the data store.

Preferably, the at least one sensor mounted externally on the subject animal comprises a motion sensor.

Preferably, the external module comprises a data processor.

Preferably, the neural transducer comprises a neural stimulator.

Preferably, the at least one active device is arranged to operate in response to an instruction.

Preferably, the instruction is based on sensed parameter data.

Preferably, the instruction is time based.

Preferably, the instruction is generated, based on sensed parameter data, by a sensor.

Preferably, the system comprises the data store, and the instruction is generated, based on sensed parameter data, by the data store.

Preferably, the instruction is received by the system from another system or a user.

Preferably, the at least one active device is arranged to operate in response to an instruction which is generated by the local computer based on sensed parameter data.

Preferably, the at least one active device is arranged to operate autonomously.

Preferably, the at least one active device is arranged to send activity data to a data store; and the data store is arranged to store the activity data in association with associated time data.

Preferably, the at least one active device comprises a neural stimulator.

Preferably, the at least one active device comprises a substance delivery device arranged to administer at least one dose of at least one substance to the subject animal.

Preferably, the substance delivery device is arranged to administer at least one of: pharmaceuticals; gene therapies.

Preferably, the substance delivery device is arranged to administer a viral vector treatment.

Preferably, the at least one active device further comprises a neural stimulator; and wherein the viral vector treatment is arranged to enable hypersensitivity or hyposensitivity to neurostimulation in at least some areas of the body of the subject animal.

Preferably, the system comprises a static part not mounted on the subject animal.

Preferably, the static part comprises at least one environmental modification device arranged for operating to produce a change in the environment experienced by the subject animal.

Preferably, the at least one environmental modification device is arranged to operate in response to an instruction.

Preferably, the instruction is based on sensed parameter data.

Preferably, the instruction is time based.

Preferably, the instruction is generated, based on sensed parameter data, by a sensor.

Preferably, the system comprises the data store, and the instruction is generated, based on the sensed parameter data, by the data store.

Preferably, the instruction is received by the system from another system or a user.

Preferably, the system comprises a static part not mounted on the subject animal and comprising at least one environmental modification device arranged for operating to produce a change in the environment experienced by the subject animal, arranged to operate in response to an instruction which is generated by the local computer based on sensed parameter data.

Preferably, the at least one environmental modification device is arranged to operate autonomously.

Preferably, the at least one environmental modification device is arranged to send activity data to a data store; and the data store is arranged to store the activity data in association with the associated time data.

Preferably, the at least one environmental modification device is arranged to provide food to the subject animal.

Preferably, the data store is part of the static part

Preferably, the system comprises a static part not mounted on the subject animal and the at least one wireless communication device is part of the static part.

Preferably, the system comprises a static part not mounted on the subject animal and the at least one local computer is part of the static part.

Preferably, the data store is arranged to store the meta data and associated time data together with the sensed neural and associated time data, and sensed parameter data and associated time data.

Preferably, the data store is arranged to enable stored neural data and parameter data to be referenced across multiple sensors or, based on time period or based on stored event data.

Preferably, the data store is arranged to store data relating to multiple subject animals and to enable stored neural data and parameter data to be referenced by subject animal.

Preferably, the system is arranged to generate and send notifications in response to the identification of predetermined events in the sensed parameter data and/or sensed neural data.

Preferably, the system is arranged to generate and send notifications in response to the identification of predetermined events in the sent activity data.

Preferably, the notifications are push notifications.

Preferably, the system further comprises a user front end arranged to enable viewing of the data stored in the data store.

Preferably, the user front end is arranged to enable the neural data and parameter data to be viewed together in a time synchronous manner.

Preferably, the user front end is arranged to enable the neural data and parameter data to be viewed in a time synchronous manner together with data regarding other events.

Preferably, the other events comprise at least one of: delivered neural stimulation; delivered treatments; other events.

Preferably, wherein the neural transducer is a neural sensor arranged for sensing neural data from nerves of the subject animal, and the system further comprises: at least one machine learning model arranged to receive neural data and to determine at least one physiological parameter value or bodily variable based upon the neural data; and a display means arranged to display data relating at least one of the neural data and the determined at least one physiological parameter value or bodily variable.

Preferably, wherein the neural transducer is a neural sensor arranged for sensing neural data from nerves of the subject animal, and the system further comprises: at least one machine learning model arranged to receive neural data and to determine at least one physiological parameter value or bodily variable based upon the neural data; and a display means arranged to display data relating to an intermediate state of the machine learning data together with at least one of the neural data and the determined at least one physiological parameter value or bodily variable.

Preferably, the display means is arranged to display data relating to an intermediate state of the machine learning data together with both the neural data and the determined at least one physiological parameter value or bodily variable.

Preferably, the system further comprises at least one sensor arranged for sensing data of a parameter of the subject animal, and the system is further arranged to calculate the at least one physiological parameter value or bodily variable based upon the data sensed by the at least one sensor; and to also display data the calculated at least one physiological parameter value or bodily variable.

Preferably, the at least one bodily variable is not directly, or not easily, measurable or is not directly, or not easily, measurable by observation of the subject animal.

Preferably, the different displayed data are synchronous.

Preferably, the display means is arranged to display data relating to an intermediate state of the machine learning data as a reduced dimensionality plot.

Preferably, the reduced dimensionality plot is at least one of: a t-SNE plot; a PCA plot; an ICA plot; or an Isomap.

Preferably, the display means is arranged to display data relating to an intermediate state of the machine learning data representing equivalence to neural populations, neural events, or neural or non-neural biomarkers, or combinations of neural and non-neural biomarkers.

Preferably, the display means is arranged to display data relating to an intermediate state of the machine learning data as vectors of classes.

Preferably, the at least one physiological parameter value or bodily variable is at least one of: heartrate of the subject animal; activity of the subject animal; temperature of the subject animal; blood glucose level of the subject animal; any vital sign of the subject animal; any physiological measurement of the whole of the subject animal, a body part of the subject animal or a sub-part of the subject animal; any data representative of a state of the whole of the subject animal, a body part of the subject animal, or a sub-part of the subject animal.

In a third aspect, the present disclosure provides a system for controlling at least one substance delivery device, the system comprising: at least one substance delivery device embedded internally within the subject animal and arranged for delivery of at least one substance to the subject animal in response to instructions from a computing device located externally of the subject animal; at least one sensor arranged for sensing data of a parameter of the subject animal and sending the sensed data to the computing device; an external module mounted externally on the subject animal and connected through a port to at least one of the at least one active device or the at least one sensor; wherein the instructions to the at least one active device are based, at least in part, on the sensed data; and wherein the through port connection comprises a sealed and ported through skin interface device.

Preferably, the through port connection is a wired connection, and preferably an electrical wired connection.

Preferably, the through port connection is a fluid or gas passage channel.

Preferably, the through port connection is an optical fiber.

Preferably, the at least one sensor is a neural sensor arranged for sensing neural data from nerves of the subject animal.

Preferably, the system further comprises: a wireless communication unit for wirelessly sending the sensed data to the computing device; means for sending the sensed parameter data to the data store; and a power supply; wherein the external module comprises at least one of the wireless communication unit and the power supply.

Preferably, the external module comprises at least one of: a data processor arranged for processing the sensed data; a data storage arranged for storing the sensed data.

Preferably, the external module comprises the computing device.

Preferably, the external module comprises the power supply.

Preferably, the system further comprises at least one wireless communication device for wirelessly receiving the sensed data from the wireless communication unit.

Preferably, the substance delivery device is arranged to administer at least one of: pharmaceuticals; gene therapies.

Preferably, the substance delivery device is arranged to administer a viral vector treatment.

Preferably, the system further comprises a neural stimulator; and wherein the viral vector treatment is arranged to enable hypersensitivity or hyposensitivity to neurostimulation in at least some areas of the body of the subject animal.

Preferably, the system comprises a static part not mounted on the subject animal and comprising the computing device.

Preferably, the skin interface device has a passageway therethrough; wherein the through port connection extends through the passageway to pass between the exterior and interior of the subject animal; and the skin interface device is arranged to maintain a homeostatic barrier.

Preferably, the skin interface device comprises a cap portion having a porous flange; wherein the porous flange is configured to receive soft tissue.

Preferably, the passageway is welded to provide a seal around the electrical connection.

In a fourth aspect, the present disclosure provides a system for processing biological data relating to a subject animal, the system comprising: at least one machine learning model arranged to receive neural data obtained from nerves of the subject animal and to determine at least one physiological parameter value or bodily variable based upon the neural data; and a display means arranged to display data relating to at least one of the neural data and the determined at least one physiological parameter value or bodily variable.

Preferably, the display means is further arranged to display an intermediate state of the machine learning data.

Preferably, the display means is arranged to display data relating to an intermediate state of the machine learning data together with both the neural data and the determined at least one physiological parameter value or bodily variable.

Preferably, the at least one bodily variable is not directly, or not easily, measurable or is not directly, or not easily, measurable by observation of the subject animal.

Preferably, the system is further arranged receive at least one physiological parameter value or bodily variable obtained by at least one sensor; and to also display data the calculated at least one physiological parameter value or bodily variable.

Preferably, the different displayed data are synchronous.

Preferably, the display means is arranged to display data relating to an intermediate state of the machine learning data as a reduced dimensionality plot.

Preferably, the reduced dimensionality plot is at least one of: a t-SNE plot ; a PCA plot; an ICA plot; or an Isomap.

Preferably, the display means is arranged to display data relating to an intermediate state of the machine learning data representing equivalence to neural populations, neural events, or neural or non-neural biomarkers, or combinations of neural and non-neural biomarkers.

Preferably, the display means is arranged to display data relating to an intermediate state of the machine learning data as vectors of classes.

Preferably, the at least one physiological parameter value or bodily variable is heartrate.

In a fifth aspect, the present disclosure provides a method of obtaining biological data from a subject animal by using a system according to the first aspect.

In a sixth aspect, the present disclosure provides a method of obtaining biological data from a subject animal by using a system according to the second aspect.

In a seventh aspect, the present disclosure provides a method of controlling a substance delivery device by using a system according to the third aspect.

In a seventh aspect, the present disclosure provides a method of processing and displaying biological data from a subject animal by using a system according to the fourth aspect.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The features of each of the above aspects and/or embodiments may be combined as appropriate, as would be apparent to the skilled person, and may be combined with any of the aspects of the invention. Indeed, the order of the embodiments and the ordering and location of the preferable features is indicative only and has no bearing on the features themselves. It is intended for each of the preferable and/or optional features to be interchangeable and/or combinable with not only all of the aspect and embodiments, but also each of preferable features.

### BRIEF DESCRIPTION OF THE DRAWINGS

Embodiments of the invention will be described, by way of example, with reference to the following drawings, in which:

FIG. 1 is a explanatory diagram of a biological data collection system according to one embodiment;

FIG. 2 is an explanatory diagram of a part of the biological data collection system of FIG. 1;

FIG. 3 is a schematic cross-sectional view of a first interface device useable in the system of FIG. 1;

FIG. 4 is a schematic cross-sectional view of a second interface device useable in the system of FIG. 1;

FIG. 5 is a more detailed schematic diagram of a part of an interface device useable in the system of FIG. 1;

FIG. 6 is a more detailed schematic diagram of a part of an interface device useable in the system of FIG. 1;

FIGS. 7a to 7e are more detailed schematic diagrams of electrical connection retaining means of an interface device useable in the system of FIG. 1;

FIG. 8 is a schematic diagram of a data handling architecture useable by the system of FIG. 1;

FIG. 9 is an example of a status monitoring and data overview screen provided by the system of FIG. 1 in operation;

FIG. 10 is an example of a data screen which may be displayed by the system of FIG. 1 in operation;

FIG. 11 is an example of another data screen which may be displayed by the system of FIG. 1 in operation; and

FIG. 12 is an example of a data screen which may be displayed by the system of FIG. 1 in operation.

Common reference numerals are used throughout the figures to indicate similar features. It should however be noted that even where reference numerals for features used throughout the figures vary, this should not be construed as non-interchangeable or distinct. Indeed, unless specified to the contrary, all features referring to similar components and/or having similar functionalities of all embodiments are interchangeable and/or combinable.

### DETAILED DESCRIPTION

Embodiments of the present invention are described below by way of example only. These examples represent the best ways of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

It should be noted that although exemplary examples, descriptions and/or embodiments are provided under separate headings, these headings should simply serve as a reading aid to provide structure to the description. For the avoidance of any doubt, the features described in any embodiment are combinable with the features of any other embodiment and/or any embodiment is combinable with any other embodiment unless express statement to the contrary is provided herein. Simply put, the features described herein are not intended to be distinct or exclusive but rather complementary and/or interchangeable.

### System Overview

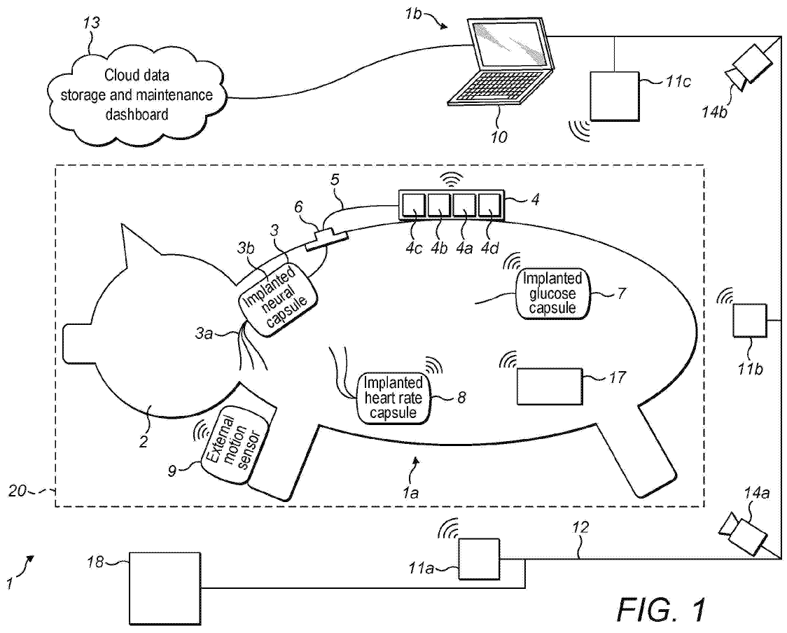

FIG. 1 shows a schematic illustration of the overall arrangement of a biological data collection system according to an exemplary embodiment. The system can also be used to control substance delivery devices.

As illustrated in FIG. 1, a biological data collection system 1 is intended to collect biological data from a subject animal 2. The system 1 may also be used to control the delivery of substances to the subject animal 2 from substance delivery devices, as will be explained below.

The collection of biological data from a subject animal 2 will generally form a part of an experiment, study, or research program of some kind, although other terminology may be used. The biological data collection system 1 is intended to collect neural data and other biological data from the subject animal 2. The biological data collection system 1 comprises a first mobile part 1a mounted on the subject animal 2 and a second static part 1b. In operation, the first mobile part 1a of the system 1 measures biological data from the subject animal 2 and transmits this wirelessly to the static part 1b of the system 1 for recording and analysis.

For the avoidance of doubt, the subject animal 2 may be a human or animal subject.

The mobile part 1a of the system 1 comprises a neural transducer module 3 implanted within the body of the subject animal 2 and an external module 4 secured to the exterior of the body of the subject animal 2. The external module 4 comprises a wireless communication unit 4a and a power supply unit 4b, and is user serviceable in operation of the system 1. The neural transducer module 3 and the external module 4 are connected by electrical connections 5 forming a wired connection between the neural transducer module 3 and the external module 4, and providing power supply connections and data communication connections between the neural transducer module 3 and the external module 4. The electrical connections 5 may comprise one or more electrical cables. In operation, the neural transducer module 3 receives electrical power from the power supply unit 4b of the external module 4 through the electrical connections 5. The neural transducer module 3 is arranged to operate as a neural sensor module 3, and measures neural activity of the subject animal 2 and sends acquired neural data derived from these measurements to the external module 4 through electrical connections 5. The wireless communication unit 4a of the external module 4 then wirelessly sends the neural data to the static part 1b of the system 1.

The mobile part 1a of the system 1 further comprises a transcutaneous device or through skin interface device 6 providing through port connection between the neural sensor module 3 implanted within the body of the subject animal 2 and the external module 4 exterior to the body of the subject animal 2. This through port connection provides a route for the electrical connections 5 to pass through the skin of the subject animal 2 to form a wired connection between the neural sensor module 3 implanted within the body of the subject animal 2 and the external module 4 exterior to the body of the subject animal 2. The through port connection of the through skin interface device 6 provides a hermetic and/or homeostatic seal for the electrical connections 5 passing between the interior and exterior of the subject animal. The through skin interface device 6 is described in more detail below.

The static part 1b of the system 1 comprises three wireless communication devices 11a to 11 c each connected to a local computer 10 by a communication network 12. The communication network 12 may be an Ethernet communication network. Each of the wireless communication devices 11a to 11c receives neural data wirelessly from the wireless communication unit 4a of the external module 4, and forwards this received neural data to the local computer 10. The local computer 10 provides data recording functionality to record biological data, such as the neural data from the signals it receives from the wireless communication devices 11a to 11c. The local computer 10 records the neural data together with time stamps applied by the local computer 10. This data recording functionality is provided by data recording software running on the local computer 10.

The local computer 10 then sends the recorded biological data and associated time data in the form of the applied time stamps to a central data storage system 13 for storage. The operation of the central data storage system 13 is described in more detail below. This recorded biological data will include the neural data, and may include other types of biological data, as will be explained below.

The mobile part 1a of the system 1 further comprises an active device 17. The active device 17 is able to cause a change to the body of the subject animal 2. The active device 17 is implanted within the body of the subject animal 2. The active device 17 is a substance delivery device comprising a substance reservoir, a controllable substance delivery system, a wireless communication device and a battery. In operation the active device 17 can respond to instructions received wirelessly from the wireless communication devices 11a to 11c of the static part 1b of the system 1 by administering doses of a substance to the subject animal 2. The amount and/or timing of the substance dose may be specified in the instructions. In some examples the active device 17 may comprise multiple substance reservoirs and may be able to administer at least one dose of a at least one substance, and optionally a plurality of different substances, to the subject animal 2 in response to the instructions. The substances may, for example, be pharmaceuticals or nutrients. The active device 17 may also respond to instructions from other sources, as will be explained below.

The local computer 10 provides control functionality to provide instructions to the active device 17. These instructions are sent by the local computer 10 through the communication network 12 to the wireless communication devices 11 to be sent wirelessly to the active device 17. This control functionality is provided by control software running on the local computer 10. The instructions sent to the active device 17 by the local computer 10 may be generated by the local computer 10 itself. Alternatively, they may be generated elsewhere and sent to the local computer 10, for example by a sensor of the system, the central data storage system 13, an external system analyzing the data recorded and/or stored by the system 1, or by a human user, such as an experimenter. In some examples the instructions sent to the active device 17 may be generated on a predetermined time basis, or may be generated based on sensed conditions of the subject animal 2, or other parameters. In some examples the instructions sent to the active device 17 may be based upon sensed neural data of the subject animal 2. In some examples the instructions sent to the active device 17 may be based upon sensed data of a parameter of the subject animal 2. This may be sensed data from any of the sensors of the system 1. In some examples the instructions may be sent to the active device 17 directly from a sensor of the system 1.

In some examples the active device 17 may comprise one or more sensors to sense conditions of the subject animal and/or a timer so that the active device 17 can operate autonomously based on a stored program or instruction set.

When the active device 17 operates, for example by administering a dose of a substance to the subject animal 2, the active device 17 sends activity data reporting the operation wirelessly to the wireless communication devices 11, which forwards this data through the communication network 12 to the local computer 10. The received activity data is then logged and time stamped by the local computer 10 and the logged activity data and associated time data in the form of the applied time stamps are sent to the central data storage system 13 for storage in the same way as the acquired biological data.

The static part 1b of the system 1 further comprises a environmental modification device 18. The environmental modification device 18 is connected to the communication network 12. In operation the environmental modification device 18 can respond to received instructions by modifying the environment of the subject animal 2. For example, the environmental modification device 18 may be able to control access to a supply of food to the subject animal 2. The type, degree and/or timing of the change may be specified in the instructions. In some examples the environmental modification device 18 may comprise multiple effectors and be able to make multiple different changes to the environment of the subject animal 2 in response to the instructions. The environmental changes may, for example, be the provision of food.

The local computer 10 provides control functionality to provide instructions to the environmental modification device 18. These instructions are sent by the local computer 10 through the communication network 12 to the environmental modification device 18. This control functionality is provided by control software running on the local computer 10. The instructions sent to the environmental modification device 18 by the local computer 10 may be generated by the local computer 10 itself. Alternatively, they may be generated elsewhere and sent to the local computer 10, for example by the central data storage system 13, an external system analyzing the data recorded and/or stored by the system 1, or by a human user, such as an experimenter. In some examples the instructions sent to the environmental modification device 18 may be generated on a predetermined time basis, or may be generated based on sensed conditions of the subject animal 2, or other parameters. In some examples the instructions sent to the environmental modification device 18 may be based upon sensed neural data of the subject animal 2. In some examples the environmental modification device 18 may comprise sensors to sense conditions of the subject animal and/or a timer so that the environmental modification device 18 can operate autonomously based on a stored program or instruction set.

When the environmental modification device 18 modifies the environment of the subject animal 2, the environmental modification device 18 sends environmental modification data reporting the modification through the communication network 12 to the local computer 10. The received environmental modification data is then logged and time stamped by the local computer 10 and the logged environmental modification data and associated time data in the form of time stamps are sent to the central data storage system 13 for storage in the same way as the acquired biological data.

In the illustrated example the subject animal 2 is located within a housing 20, such as a cage or enclosure. The housing 20 limits the area over which the subject animal 2 can move, but is sized and shaped to allow the subject animal 2 sufficient freedom of movement to allow natural behavior of the subject animal 2. It will be understood that the size and shape of the housing 20 may be selected in any specific application of the system 1 based on factors including the species of the subject animal 2 and the nature of the biological data to be collected. The housing 20 may, for example, ensure that the subject animal 20 remains localized within an area where reliable wireless communication can be maintained between the mobile part 1a and the static part 1b of the system 1.

In the illustrated example the wireless communication devices 11a to 11c are located at spaced apart positions around the housing 20 to ensure that at least one of the wireless communication devices 11a to 11c can maintain reliable wireless communication with the mobile part 1a of the system 1, such as the external module 4.

The neural sensor module 3 comprises a plurality of sensor electrodes 3a located adjacent to nerves of the subject animal 2 and a data collection capsule 3b. The sensor electrodes 3a may, for example, each comprise one or more sensor cuffs extending around one or more nerves in order to convert changes in the electrical state of the nerves into electrical signals on the sensor electrodes 3a. The data collection capsule 3b comprises electrical circuitry for sensing the electrical signals on the sensor electrodes 3a and converting them into neural data in a suitable format for transmission to the external module 4 along the electrical connections 5. Typically the electrical circuitry of the data collection capsule 3b will comprise amplifiers and analogue to digital converters, together with other components. In some examples the neural sensor module 3 may comprise optical sensors in place of, or in addition to, the sensor electrodes 3a.

The sensor electrodes 3a may comprise a shape, material and/or particular properties, mechanical or otherwise, which are biocompatible and minimize tissue reaction. Additionally, the sensor electrodes 3a may be selected to minimize tissue damage caused from chemical reactions, toxicity or otherwise. In some examples the sensor electrodes 3a may comprise other types of suitable electrodes including needle, sieve or micro array electrodes and/or implantable myoelectric sensors or similar, in place of, or in addition to, sensor cuffs.

The sensor electrodes 3a that are located adjacent to the nerves may be placed located or sheathed in such a way as that electrodes-nerves construct is protected or isolated from external forces, motion, surrounding signals and noise signals. In some examples protection or isolation is achieved by biological tissues, for instance, inside bone, under periosteum, in muscle. In other examples protection or isolation is achieved inside engineered materials, for instance, inside or under a metal implant, plastic implant or other substructure created for the purpose, this could include solid implant materials or biological or nonbiological glues, resins or other materials that can be deployed around the sensor electrode site. For instance, tisseal (or other fibrogen based glues and sealants), silicon, cyanoacrylate, or otherwise.

As discussed above, the external module 4 comprises a wireless communication unit 4a and a power supply unit 4b. The power supply unit 4b may comprise a power store, such as a battery. The wireless communication unit 4a is arranged to receive data from the data collection capsule 3b along the electrical connections 5, and to wirelessly transmit this received data to the wireless communication devices 11a to 11c of the static part 1b of the system 1. The power supply unit 4b is arranged to provide electrical power to the wireless communication unit 4a, and also to provide electrical power to the data collection capsule 3b along the electrical connections 5. In some examples the external module 4 may additionally comprise a data processing unit 4c to carry out some processing of the received data before transmitting it and/or a data storage unit 4d to store the received, and possibly processed, data temporarily. The data storage unit 4d may, for example be used as a local data storage buffer by the mobile part 1a of the system 1 in the event of any loss of wireless connectivity with the static part 1a of the system 1.

It will be understood that because the power supply unit 4b is not implanted, and is located outside the body of the subject animal 2, the physical size, and thus the power capacity, of the power supply unit 4b may be much larger than would be practical in an implanted device. Further, the power supply unit 4b can be accessed for recharging/refueling or replacement as desired. Accordingly, the operating lifetime of the neural sensor module 3 can be extended indefinitely without any requirement for tethering.

In the illustrated embodiment the external module 4 comprises a wireless communication unit 4a and a power supply unit 4b, and may optionally also comprise a data processing unit 4c and/or a data storage unit 4d, In other examples the external module 4 may comprise different components.

In one alternative arrangement the external module 4 may comprise a power supply unit 4b only, without any wireless communication unit 4a. In this example the neural sensor module 3 may be provided with a wireless communication unit. Such a wireless communication unit may be comprised in, or associated with, the data collection capsule 3a. In another alternative arrangement the external module 4 may comprise a wireless communication unit 4a only, without any power supply unit 4b. In this example the neural sensor module 3 may be provided with a power supply. Such a power supply may be comprised in, or associated with, the data collection capsule 3a. In either of these examples the external module 4 may additionally comprise a data processing unit 4c and/or a data storage unit 4d.

In one example the data processing unit 4c of the external module 4 may generate instructions to the active device based on the acquired neural data.

In another alternative arrangement the external module 4 may comprise a power supply unit 4b and the neural sensor module 3 may also be provided with a power supply. In this arrangement the transfer of power between the external module 4 and the neural sensor module 3 may, or may not, be supported.

In general, the required power supply, wireless communication, data processing, and data storage functionality of the mobile part 1a of the system 1 can be split between the external module 4 and system components implanted within the body of the subject animal 2, such as the neural sensor module 3, as preferred in any specific implementation.

As explained above the transcutaneous device or through skin interface device 6 provides a hermetic and/or homeostatic seal for the electrical connections 5 passing between the interior and exterior of the subject animal. This provides a stable long-term interface between the intracorporeal and extracorporeal environments, and prevents infection or other medical problems which could impact the value of the gathered data and/or harm the subject animal.

In the illustrated example only a single neural sensor module 3 is shown, for clarity. In practice there may be a plurality of neural sensor modules 3 connected to the external module 4. The different ones of the plurality of neural sensor modules 3 may sense neural data from different locations in the body of the subject animal 2. In examples having a plurality of neural sensor modules 3 these may be the same, or different. In examples where some of the power supply, wireless communication, data processing, and data storage functionality of the mobile part 1a of the system 1 is located at the neural sensor modules 3, the different neural sensor modules 3 may provide different functionalities.

As shown in FIG. 1, the mobile part 1a of the system 1 further comprises a number of further sensor modules in addition to the neural sensor module(s) 3. In the illustrated embodiment these further sensor modules comprise a glucose sensor module 7, a heart rate sensor module 8, and a motion sensor module 9. In other examples other types of sensors may alternatively or additionally be used.

The glucose sensor module 7 is implanted within the body of the subject animal 2 and comprises a blood glucose level sensor, a wireless communication device, and a battery. In operation the glucose sensor module 7 measures blood glucose levels and sends the resulting blood glucose data wirelessly to the wireless communication devices 11a to 11c of the static part 1b of the system 1.

The heart rate sensor module 8 is implanted within the body of the subject animal 2 and comprises a heart rate sensor, a wireless communication device, and a battery. In operation the heart rate sensor module 8 measures heart rate and sends the resulting heart rate data wirelessly to the wireless communication devices 11a to 11c of the static part 1b of the system 1.

The motion sensor module 9 is attached to the exterior of the body of the subject animal 2 and comprises a motion sensor, a wireless communication device, and a battery. In operation the motion sensor module 9 measures movement of the subject animal 2 and sends movement data wirelessly to the wireless communication devices 11a to 11c of the static part 1b of the system 1. The motion sensor module 9 is attached to a limb of the subject animal 2 and measures movement of that limb. In some examples multiple motion sensor modules may be used to measure the movement of different limbs of the subject animal 2, and/or movement of the subject animal 2 as a whole. The motion sensor module 9 may, for example, be an inertial measurement unit (IMU).

As has been discussed above, the power demands associated with the neural sensor module 3 may be higher than the power demands of the further sensor modules because of the requirement to collect a very large volume of neural data at a high level of fidelity, and the low voltage levels and high data rates of the sensed nerve activity. As a result, the batteries of the implanted further sensor modules, such as the glucose sensor module 7 and the heart rate sensor module 8, may be able to support a sufficient operating lifetime to allow long term monitoring of the subject animal 2. However, If desired one, some, or all of the further sensor modules could be connected to the external module 4 by the electrical connections 5 to allow these further sensor modules to be supplied with power by the power storage unit 4b of the external module 4.

The data recording software running on the local computer 10 records biological data relating to blood glucose levels, heart rate and movement of the subject animal 2 from the data signals it receives from the wireless communication devices 11a to 11c which originate from the glucose sensor module 7, heart rate sensor module 8 and motion sensor module 9 respectively, and time stamps this data, in the same way that the local computer 10 records and time stamps neural data, as discussed above. The local computer 10 then sends this recorded biological data and associated time data in the form of the applied time stamps to the central data storage system 13 for storage.

The examples of further sensor modules discussed above of a glucose sensor module, a heart rate sensor module, and a motion sensor module are exemplary only. Some further examples of types of further sensors which could be alternatively or additionally used in the system 1 comprise one, some, or all of: sensors for peripheral or central nerve recording; ElectroCardioGram (ECG) sensors; Electro CortigoGram (ECoG) sensors; Electro Muscular Graph (EMG) sensors; blood pressure sensors, such as vascular pressure sensors; airway pressure sensors; intrapleural pressure sensors; gastric activity sensors; gastric PH sensors; bladder pressure and/or state sensors; Inertial Measurement Units (IMU); muscle activation sensors; chemical sensors, such as drug or medication sensors, and still or video cameras mounted internally or externally to the body of the subject animal. This listing of possible sensors is not intended to be exhaustive.

FIG. 1 shows the mobile part 1a of the system 1 comprising an active device which is a substance delivery device 17. The mobile part 1a of the system 1 may additionally or alternatively comprise other active devices. The active devices may be any device which can cause a measureable change in the subject animal 2. Examples of possible active devices which could be used in the system 1 comprise one, some, or all of: peripheral or central neural stimulation devices; devices that dispense nutrients; devices that dispense pharmaceuticals; devices that administer gene therapies, for example CRISPR; devices that administer viral vector treatments. This listing of possible active devices is not intended to be exhaustive.

The active devices may be implanted inside the body of the subject animal 2, or they may be located external of the body of the subject animal 2, as appropriate. The active devices may be instructed to operate, or may operate autonomously in a similar manner to the active device 17. In some examples an active device may comprise one or more sensors to sense the conditions of the subject animal so that the active device can operate autonomously.

In some examples the neural transducer module 3 may be arranged to act as an active device in addition to, acting as a sensor, allowing the neural transducer module 3 to act as a two way neural interface. In such examples the neural transducer module 3 maybe able to electrically stimulate nerves in addition to measuring electrical activity on nerves. This capability may be useful to carry out nerve stimulation and measure the resulting neural response in order to assess nerve conduction health.

In some examples the neural transducer module 3 may be arranged to act as an active device instead of acting as a sensor, allowing the neural transducer module 3 to act as a nerve stimulator. In such examples the neural transducer module 3 maybe able to electrically stimulate nerves This capability may be useful to carry out nerve stimulation in order to assess nerve conduction health.

The use of active devices in the mobile part 1a of the system 1 is not essential. In some examples the mobile part 1a of the system 1 may only comprise sensors.

FIG. 1 shows the static part 1a of the system 1 comprising an environmental modification device 18 which provides food. The static part 1a of the system 1 may additionally or alternatively comprise other environmental modification devices which modify the ambient environment experienced by the subject animal 2. Examples of possible environmental modification devices which could be used in the system 1 comprise one, some, or all of: devices that dispense food; devices that changes ambient light levels; devices that change ambient temperature. This listing of possible environmental modification devices is not intended to be exhaustive.

The environmental modification devices may be instructed to operate, or may operate autonomously in a similar manner to the environmental modification device 18. In some examples an environmental modification device may comprise one or more sensors to sense the conditions of the subject animal so that the environmental modification device can operate autonomously.

As shown in FIG. 1, the static part 1b of the system 1 further comprises one or more video cameras 14 connected to the local computer 10 by the communication network 12. In the illustrated embodiment of FIG. 1 the static part 1b of the system 1 comprises two video cameras 14a and 14b. It will be understood that this is not essential and that a single video camera 14 or more than two video cameras 14 may be used if desired.

The video cameras 14a and 14b are arranged so that they each have a field of view including the entire area over which the subject animal 2 can move, as defined by the housing 20. The video cameras 14a and 14b are arranged to view the housing 20 and subject animal 2 from different angles. The use of multiple video cameras viewing from different angles ensures that all relevant activity of the subject animal is visible to at least one video camera. Further, the use of multiple video cameras viewing from different angles may enable more accurate determination of the movement and/or orientation of the subject animal 2 and/or its appendages.

The video signals from the video cameras 14a and 14b are supplied to the local computer 10. The local computer 10 then time stamps the received video data and sends this acquired video data together with associated time data in the form of the applied time stamps to the central data storage system 13 for storage. The associated time data may be generated by the video cameras 14, or by the local computer 10.

The housing 20 is formed in part, or entirely, of a transparent plastics material in order to prevent the housing 20 blocking the fields of view of the video cameras 14, and to minimize the risk of the housing 20 interfering with wireless communication between different parts of the system 1. Such interference with wireless communication could, for example, be caused by metal parts of the housing.

### Electrical Connection Structure

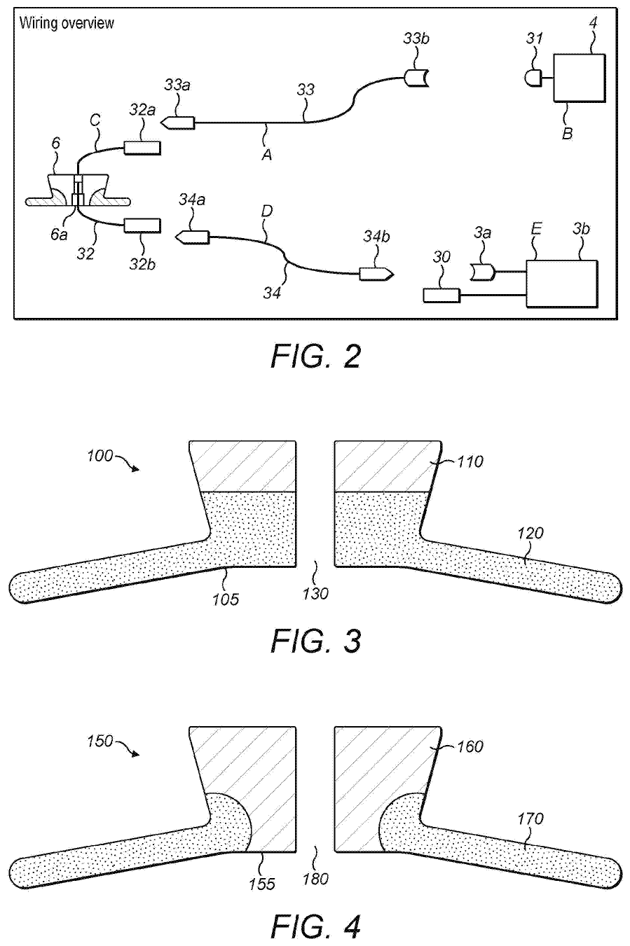

FIG. 2 shows a schematic illustration of the arrangement of the electrical connections 5 according to the exemplary embodiment.

The neural sensor module 3 implanted within the body of the subject animal 2 comprises an electrical connector 30. The external module 4 located exterior to the body of the subject animal 2 comprises an electrical connector 31.

The electrical connections 5 comprise a bridging section 32 having a pair of electrical connectors 32a, 32b at its ends. The bridging section 32 passes through an access port 6a in the transcutaneous device or through skin interface device 6 to form an electrical connection between the interior and exterior of the body of the subject animal and provide a sealed barrier between the interior of the body of the subject animal and the external environment, so that the port forms a homeostatic seal.

The electrical connections further comprise an external section 33 having a pair of electrical connectors 33a, 33b at its ends, and an internal section 34 having a pair of electrical connectors 34a, 34b at its ends.

In order to form the electrical connections 5 between the neural sensor module 3 and the external module 4, the connector 33b of the external section 33 is linked to the connector 31 of the external module 4, the connector 33a of the external section 33 is linked to the connector 32a of the bridging section 32, the connector 32a of the bridging section 32 is linked to the connector 34a of the internal section 34, and the connector 34a of the internal section 34 is linked to the connector 30 of the neural sensor module 3.

The different electrical connectors may be any suitable type of electrical connector. Many types of electrical connector are known to the skilled person in the technical field of the present invention, and accordingly it is not necessary to describe the electrical connectors in detail herein. In some examples the electrical connections which are located internally of the body of the subject animal 2 in use may be different from the electrical connections which are located externally of the body of the subject animal 2 in use.

In use of the system 1, during implantation of the plurality of sensor electrodes 3a, the data collection capsule 3b, and the through skin interface device 6, these components and the external module 4 can be located at optimal biological positions inside and outside of the body of the subject animal 2. The lengths of the external section 33 and internal section 34 of the electrical connections 5 may be selected as required for any specific positioning and geometry selected for the different components and the routes of the electrical connectors 5 between them. The internal section 34 of the electrical connections 5 can be passed between locations within the body of the subject animal 2 using tunneling or catheter passing instruments. In some examples tunneling and catheter passing instruments may both be used on different electrical connections 5. In some examples optimum pathing may be to tunnel the electrical connectors 5 subdermally in order to avoid affecting other bodily functions.

In the illustrated example only a single electrical connections 5 to a single neural sensor module 3 is shown, for clarity. In practice there may be a plurality of neural sensor modules 3 connected to the external module 4 by a plurality of electrical connections 5. In some examples the electrical connections 5 may include junctions or branches.

In examples where there are a plurality of electrical connections 5 these may comprise a plurality of bridging sections 32 which all pass through a single common port of the through skin interface device 6. Alternatively, the plurality of bridging sections may pass through a plurality of separate ports of the through skin interface device 6.

In the illustrated example of FIG. 2 the bridging section 32 is shown relatively long with ends extending away from the through skin interface device 6. This is not essential. The bridging section 32 may be of any convenient length, and does not need to extend the same length on each side of the through skin interface device 6. In some examples the length of the bridging section may be substantially the same as the thickness of the through skin interface device 6, so that the electrical connectors 32a, 32b effectively project from the inner and outer surfaces of the through skin interface device 6. In some examples the electrical connectors 32a, 32b may be attached to the through skin interface device 6 to improve strength and stability. In some examples the connectors 32a and 32b may be recessed into surfaces of the through skin interface device 6 to form sockets to receive the connectors 33a and 34a.

### Through Skin Interface Device

FIGS. 3 and 4 are cross-sectional views of interface devices suitable for use as the transcutaneous device or through skin interface device 6 according to exemplary embodiments. The interface devices of these embodiments are designed to maintain a homeostatic barrier between the interior of the body of the subject animal 2 and the external environment.

FIG. 3 is cross-sectional view of an interface device according one embodiment. The interface device 100 of this embodiment is suitable for integration with soft tissue, for example skin. The interface device 100 comprises a cap portion 110 and a surrounding flange 120. In this embodiment, the cap portion 110 and surrounding flange 120 are substantially non-planar, specifically, the cap portion 110 is raised from the surrounding flange 120. It will however be appreciated that the surrounding flange can be substantially planar to and/or extend along a similar path as the cap portion. For example, the surrounding flange can extend substantially from the side of the cap portion 110, preferably such that the surrounding flange 120 is substantially flush with the side of and/or extends along a common path to the cap portion 110. For example, the interface device 100 can be relatively flat and uniformly round, e.g. disc shaped. In alternative embodiments, for example as depicted, the flange 120 may protrude at a downwards trajectory, e.g. extending from the side of the cap portion 110 at an angle. The flange 120 may be integral with the cap 110 or may be distinct from but fixable thereto.

Preferably, the flange 120 is designed to allow the skin of the subject animal 2 to grow into it. This configuration enables the homeostatic barrier between internal and external surfaces of the body/animal that is normally provided by the skin to be maintained. In some preferable embodiments, in use, the skin (or other soft tissue) is extended along the length of the flange 120 such that the leading edge of the skin abuts the cap portion 110. It will be appreciated that the dimensions of the surrounding flange may be adaptable. The adaptable dimensions may include one or more of the angle that the flange protrudes in respect of the cap portion, the geometry of connection between the flange and the cap portion including but not limited to the curvature radii of the connection between the flange and the cap portion, the relative sizing of profile of the cross-section of the flange, the length of the flange and the thickness of the flange.

Preferably, the geometry of the flange 120 of the cap portion 110 may be designed to promote soft tissue ingrowth, soft tissue adherence and to minimise stress concentrations (and maximize interface strength) at the skin/device interface when in use (it is also preferably designed to allow long term nutrient supply to the tissues on the outside of the flange 120 so they can maintain long term health.

For example, the thickness of the flange 120 may be substantially uniform across its length or may vary as will be described in more detail below. In the present embodiment, the thickness of the flange 120 is substantially uniform across its length with the periphery of the flange ending in a tapered/rounded manner. It will however be appreciated that the different configurations may be provided additionally or alternatively. For example, the thickness of the flange might be tapered across its length so as to arrive at the periphery at a point which may be rounded or pointed.

In the embodiment depicted in FIG. 3, the entirety of the flange and the lower surface 105 (the surface facing the subject animal 2 in use) of the cap portion 110 is porous. In this embodiment, the cap portion 110 of the interface device 100 comprises a substantially solid disk shape with a specified thickness as its upper portion and a lower or interior surface 105 which is porous. Advantageously, by providing a flange 120 which is constructed of an open cell porous material, this enables soft tissue, for example skin tissue to grow into it. Additionally, by providing porous material on the lower surface 105 of the cap portion 110, this enables soft issue, for example muscle tissue, to grow into this interior surface 105 of the interface device 100 as well.

Although in this embodiment, the entirety of the flange and lower surface of the cap comprises a porous material, it will be appreciated that this is not required and only a portion or portions of porosity may be required to achieve the same effect. Not wishing to be bound by theory and as will be discussed in more detail below, it is believed that the more porous the material the more nutrient transfer etc. is facilitated; the flipside being that the more porous the material the weaker the structure. The holes are of the porous material are preferably smooth to avoid damage to sutures and/or nutrients flowing through; for example by forming transport capillaries. In the present embodiment, the porous material along the lower surface 105 of the interface device extends along the flange 120/cap portion 110 such that when the flange receives soft tissue, particularly skin, in use, the soft tissue/skin contacts porous material not only along its inwardly edge, but also at its leading edge.

It will also be appreciated that in alternative embodiments, the cap portion 110 need not comprise a solid disk shape and/or cap shape, but rather could have a rounded shape or any other shape providing some free surface area and allowing attachment to the surrounding flange 120.

In the alternative embodiment illustrated in FIG. 4, although the geometrical dimensions of the interface device 150 are substantially congruent to the dimensions of the interface device 100 of FIG. 3, it will be noted that porous material in this embodiment does not extend along the entire lower or inside surface 155 (surface facing the subject animal 2 in use) of the interface device 150. Instead, the entirety of the flange 170 in this embodiment is porous, as described in respect of FIG. 3, but only a portion of the inside/lower surface 155 of the cap portion 160 is porous. Preferably, and as is depicted, the porous material extends along the flange 170 as well as a portion of the cap portion 160 so that when in use the soft tissue extends along the flange to abut the periphery of the cap portion 160. With this configuration, the leading edge of the skin/soft tissue abuts a porous portion such that the skin/soft tissue can integrate with the porous material on two sides, at the leading edge as well as underneath. Advantageously, the disclosed arrangement affords the benefit of ensuring that all edges/surfaces of the interface device 150 which are in contact with the soft tissue (particularly skin) comprise a porous material, whilst increasing the structural integrity of the interface device 150 and ability to maintain the homeostatic barrier by maintaining a solid or partially solid (e.g. non-porous) core. It will be appreciated that the depicted illustration provides the aforementioned preferable advantage but that alternative arrangements could be envisaged which achieved a substantially similar effect, some such alternative designs being exemplified below.

Although the entirety of the lower surface 155 of the interface device 150 does not comprise porous material, it will be appreciated that this described embodiment still discloses that a majority of the surface area its lower surface 155 comprises porous material. Without wishing to be bound by theory, it is envisaged that the greater the proportion of surface area contact between the lower surface 155 of the interface device 150, the more soft tissue integration during use.

Advantages of the aforementioned embodiments, namely where the biological tissue abuts the inner surface of the cap portion, include a minimization of the space between the interface device 100, 150 and the soft tissue. Without wishing to be bound by theory, it is believed such minimization reduces the risk of infection, edema or internal tissue necrosis.

Preferably, this surface design of the interface device 100, 150 comprises a pore size between 200-300 μm which, although not wishing to be bound by theory, is based on field wide tissue engineering knowledge of the acceptable range of pore sizes that are viable for cell health. In one exemplary embodiment, the flange 120, 170 comprises a skin compatible surface which may contain pores of the appropriate size further defined herein (for example 200 μm), and a density with a lower bound pore density of 1/mm³ and an upper bound inferred by the pore size.

The interface devices 100, 150 are configured for soft tissue integration. For example, the surrounding flange 120, 170 is configurable to receive soft tissue; for example, the skin of a patient. Advantageously, the interface device 100, 150 in these embodiments, provides mechanical, neural and/or soft tissue integration with a subject animal.

In both FIGS. 3 and 4, an access port 130, 180 is disclosed which extends though the interface device 100, 150 to provide a channel or conduit there through. In FIG. 3, the exemplified port 130, extends though the cap portion 110 as well as the flange 120 whereas in view of the alternative construction of the interface device 150 of FIG. 4, the port 180 extends through only the cap portion 160.

When the interface devices of FIGS. 3 and 4 are used as the transcutaneous device or through skin interface device 6 the access port 130, 180 is the opening in the through skin interface device 6 through which the electrical connections 5, and specifically the bridging section 32, passes. As discussed above with reference to FIG. 2, the access port 130, 180 is sealed around the electrical connections 5 to provide a port forming a homeostatic seal.

Although the access port 130, 180 in these embodiments has a fixed dimension and are located at a predefined position in respect of the interface device 100, 150, namely having a uniform substantially cylindrical shape and extending through substantially the central axis of the interface device 100, 150, this is only exemplary. For example, the port 130, 180 may in fact comprise one or more ports, wherein any one or more of the ports comprise any dimension and shape, uniform or not, and be positioned anywhere on the interface device 100, 150.

As is discussed above, in some examples there may be additional ports, with different electrical connections passing through different ports.

In addition to providing a passageway for the electrical connections, the one or more access ports 130, 180 may comprise one or more of the following alternative or additional functions:

Passage of biosensors for detecting biofilm formation, edema or other conditions;

Passage of cables, wires or electrical contacts carrying electrical data for control of any other implanted device; equally to allow replacement and/or upgrade of the electronics with minimal disturbance to the body, for example reducing the necessity of major surgery;

An aperture through which fluids or gasses can be passed either continuously, periodically or in a single instance either through the port directly or through a conduit that passes through the port for purposes including the promotion and maintenance of tissue health by mechanical stimulation, nutrient flow or other means.

An aperture through which an optical fiber or cable can be passed.

Access for surgical procedures including keyhole surgery, this may include to remove, update, replace and/or reposition internal components of the system, for example, the sensor cuff.

Access for other medical procedures including but not limited to administering of medicines, draining of edema fluid or care of internal tissues.

The ports 130, 180 can be adapted over time. For example, the ports 130, 180 can be configured to be sealed when implanted, but can be modified in used to open the ports 130, 180 to allow for access as required.

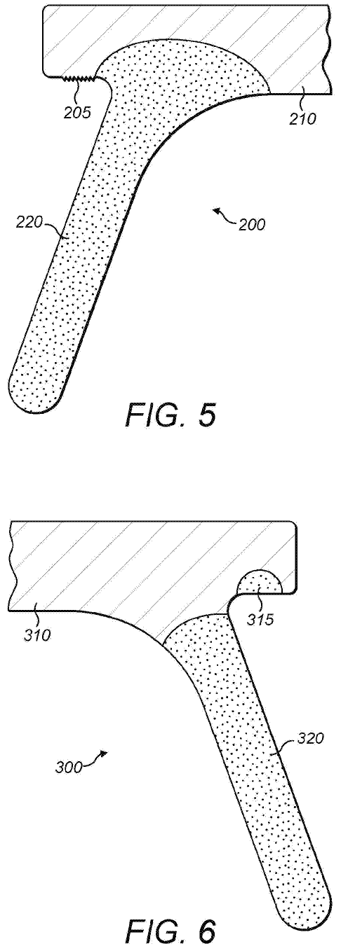

FIG. 5 is a cross-sectional view of another interface device suitable for use as the transcutaneous device or through skin interface device 6 according to exemplary embodiments. In this exemplary embodiment, the interface device 200 comprises a cap 210 and a surrounding flange 220. The cap portion 210 in this embodiment is substantially planar and comprises a substantially solid material. However, it will be appreciated that this need not be the case and the cap could have a curved or domed shape nor need the entire portion comprise a solid material. Indeed, it will be appreciated that the some or all of the properties, dimensions and/or features of the interface device 100, 150 described above could be applied in respect of the present embodiment. In particular, the cap portion 210 comprises an access port, which is not visible in FIG. 5.

The surrounding flange 220 of this embodiment extends substantially downwards from the cap portion 210 and is substantially porous. In this embodiment, the flange 220 extends from the cap portion 210 specifically extending from a predefined distance from the periphery or outer edge of the cap 210. The spacing is such that the cap portion 210 extends beyond the point at which the flange 220 engages with or attaches the cap 210, thereby providing a lip or cover. In use, this lip or cover serves as a mechanical or physical means for protecting the soft tissue engaged with the flange 220; for example, to prevent accidental pulling, tension, pressure or otherwise on this area and particularly in respect of the leading edge of the soft tissue.

The interface devices 100, 150, 200 comprise a bio-compatible material. It will be appreciated that it is not essential for the entirety of the interface device or the cap portion to consist of a bio-compatible material, but rather it is preferable that any edges and/or surfaces which in contact with the skin, vascular or muscular tissue of the patient consist substantially thereof. As such, in some embodiments only the surrounding flange or a part thereof and/or the inner surface of the cap portion (i.e. the side of the cap facing the patient in use) or a part thereof may comprise the bio-compatible material. Therefore, although the entire interface device 100, 150, 200 may consist of a bio-compatible material, e.g. only parts of the cap portion in contact with biological tissue will comprise bio-compatible materials. For example, the flange 220 (or any part there of) and/or a part(s) of the cap portion may comprise titanium (alloys thereof including Ti6Al4V), stainless steel (and its derivatives), for example having SAE grade 316, high-density, or ultra-high-density polyethylene (HDPE or UHDPE), polylactic acid (PLA), polypropylene (PP) or other FDA-approved polymer or metal, and/or combinations or mixtures thereof.

The cap portion 210 may comprise a flexible material, for example, but not limited to a polymer which may or may not be coated.

In this embodiment, the edges of the cap portion 210, specifically where the cap portion 210 meets the flange, are concave for skin integration. This arrangement addresses the need to ensure maintenance of homeostatic barrier which avoids/prevents infection. However, it will be understood that other dimensions, shapes or specifications may be provided.

As mentioned above, the parameters of the flange 220 which may be customizable including the following: the angle (θ), length (I), flange thickness, geometry of the interface surface, number size and location of cross flange holes, pore structure (size and density), curvature radii and/or relative sizing of sections of the overall profile of the flange cross-section.

Although in this example, the flange 220 is at a 45 deg angle to the cap 210, the flange 220 may be angled at any degree, for example at or less than 90 deg, at or less than 80 deg, at or less than 70 deg, at or less than 60 deg, at or less than 50 deg, at or less than 40 deg, at or less than 30 deg, at or less than 20 deg, at or less than 10 deg or any intermediate thereof.

Although the flange 220 in this embodiment is exemplified as being 15 mm long and 3 mm thick, the flange may be any length including 5 mm, 10 mm, 20 mm, 25 mm, 30 mm to 35 mm or any intermediate thereof and may be any thickness ranging from less than 1 mm, less than 2 mm, less than 3 mm, less than 4 mm to less than 5 mm.

As shown in FIG. 5, an lip or rim 205 is formed at the spaced distance (e.g. between the edge or periphery of the cap portion and the point the flange connects or attached the cap). This lip or rim 205 preferably comprises, at least a portion having a smooth finish. Such finish can be achieved for example during a machining process. It will be appreciated that alternative methods of manufacture may be used.

Although in this embodiment, only a portion of the lower surface of the rim or lip 205 comprises a smooth finish, it will be appreciated that any amount of the entire lower surface of the rim or lip 205 can comprise such material for this purpose.

It will also be appreciated that the aforementioned preferable arrangement is equally applicable to an interface device, such as those described in respect of FIGS. 3 and 4.

It will be appreciated that the radius and/or the surface area of the interface device, specifically the cap portion can be any dimension. The cap portion of the interface device in this embodiment or otherwise need not comprise a disk shape, but instead may comprise a (substantially or semi) conical, (substantially or semi) oblong or any other desired configuration. Furthermore, the cap portion, in this embodiment or otherwise, may or may not be rotationally symmetric.

Not wishing to be bound by theory, it is believed that the larger the surface area of the cap portion, the less chance of any infection around the skin integration area where the flange adjoins the skin of the subject animal 2, spreading along the electrical connections 5 within the body of the subject animal; i.e. the more distance between these two features the less chance of infection spreading. Therefore, in one preferred embodiment, the radius of the cap portion 210 is not less than 1 cm.

The flange preferably comprises a biocompatible material, and more preferably a biomimetic surface microstructure as will be described in more detail below. In one additional or alternative embodiment, the material may include porosity at surface, open-celled foam bulk structure, possibility of through-surface pores. Pore sizes may be in the range of 50 μm to 800 μm. It will appreciated that the pore size may or may not be uniform and/or the porosity may extend any part or substantially all of the flange and/or cap portion. In some embodiments, the pore sizes range from 100 μm to 750 μm, 150 μm to 700 μm, 200 μm to 650 μm, 250 μm to 600 μm, 300 μm to 550 μm, 350 μm to 500 μm, 400 μm to 450 μm or any combined or intermediate range thereof.

Not wishing to be bound by theory, it is believed that below the lower limit of 50 μm, cells that penetrate are unlikely to survive due to restricted space and lack of nutrients, and above the upper limit of 800 μm the strength of the mechanical junction may decrease. The flange may be designed to be porous through substantially its full thickness with the open cell structure.

The porosity of the flange 220 comprises a varying porosity which ranges from a more porous region where the flange 220 contacts the cap 210 to a less porous region at its periphery. It will be appreciated that the porosity could increase gradually across the length of the periphery or alternatively there could be distinct portions or zones across the length with different porosities. Not wishing to be bound by theory, it is believe that having an increased porosity in the region adjacent to the leading edge of the soft tissue when the device is in use will enhance integration and therefore reduce subsidence.

In some preferable embodiments, the flange 220 and/or any other feature comprising a porous material may comprise hydroxyapatite and/or any other material which promotes growth and/or integration of tissue groups.

FIG. 6 is a cross-sectional view of a through skin interface device according to one embodiment. The interface device 300 of this embodiment comprises each of the features of the interface device 200 described above in respect of FIG. 5, but for the extent to which the porosity extends along a cap portion 310. As has been mentioned above, it is desirable that each of the surfaces of the interface device which engage with soft tissue are porous. However, it will also be appreciated that introducing porosity also reduces the mechanical strength and stability of the overall structure of the interface device. Therefore, in this embodiment, the porosity extends substantially the length of flange 320. The porosity of the flange 320 can be uniform or non-uniform, as described above.

In this embodiment, a further porous portion 315 is provided in the cap 310. In this embodiment, the porous portion 315 is separate or spaced as a distance from, e.g. non-adjacent to, the porous flange 320, i.e. the porous portions are non-contiguous. The porous portion 315 is positioned/arranged on the cap 310 such that in use it is arranged to receive or engage with the leading edge of the soft tissue. Preferably therefore, the sizing and location of the porous portion 315 is such that it facilitates engagement with the soft tissue whilst ensuring spacing between it and the flange 320.

It is believed that by provided spacing or a break between the porous flange and the porous portion (e.g. on the cap) receiving the soft tissue/skin, there is a reducing in the risk of infection caused at the leading edge spreading through the flange whilst maintaining the benefits afforded by embodiments where the soft tissue/skin engages porous material at all surfaces.

Although in this embodiment the porosity is substantially uniform, it will be appreciated that the porosity of the flange 320 and/or the porous portion 315 can have different, non-uniform and/or varying porosities. Furthermore, it should be noted that the specification made in respect of the porous potion 315 of this embodiment is equally applicable to general soft tissue interface devices such as devices 100, 150 as described in FIGS. 3 and 4.

In some examples the ports through the interface devices may comprise threaded bores. This may simplify manufacture of the ports simplified in that a standard drill can be used to produce the ports.

In some examples the ports may form a substantially cylindrical channel with a flange or rim around the lower or inner most surface of the interface device. Such a channel will have a wider opening towards the soft tissue of the subject animal than the outward opening.

In some examples the ports could be substantially frusto-conical in shape. It is envisaged that by providing a larger surface area of the port adjacent to the subject animal, the port facilitates a great degree and area of access when in use

In some examples the ports may comprise sheathing, for example a plastic material placed in the channel formed by the ports to facilitate ease of use and/or to provide an additional layer to ensure the homeostatic barrier formed by the interface device is substantially intact. For example, the sheathing could be provided with antiseptics or analgesics or otherwise and/or could be positioned during surgery. In some embodiments, this sheathing can be replaceable and/or changeable.

In some examples the ports may extend through the cap and flange of the interface device. The size and shape of the ports need not be uniform or similar nor do they need to be circular, creating a substantially cylindrical shaped channel though the interface device. It will be appreciated that one or more of the ports can have any desired shape or dimension.

In some examples the port through the interface device may be sealed around the electrical connections 5 by welding.

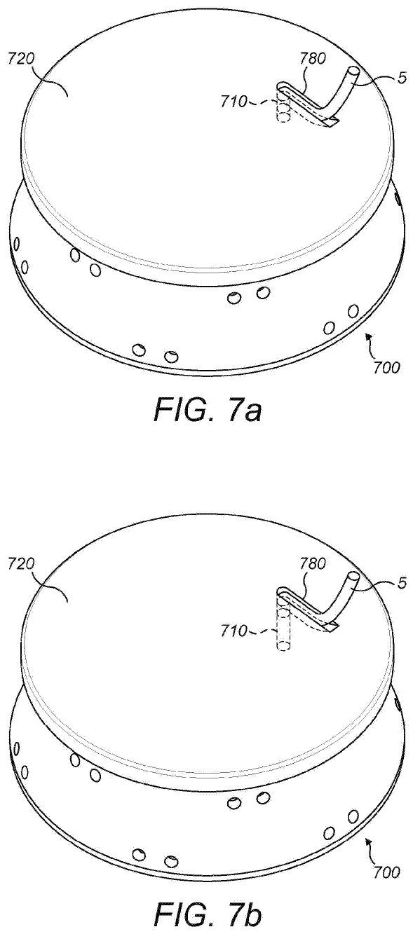

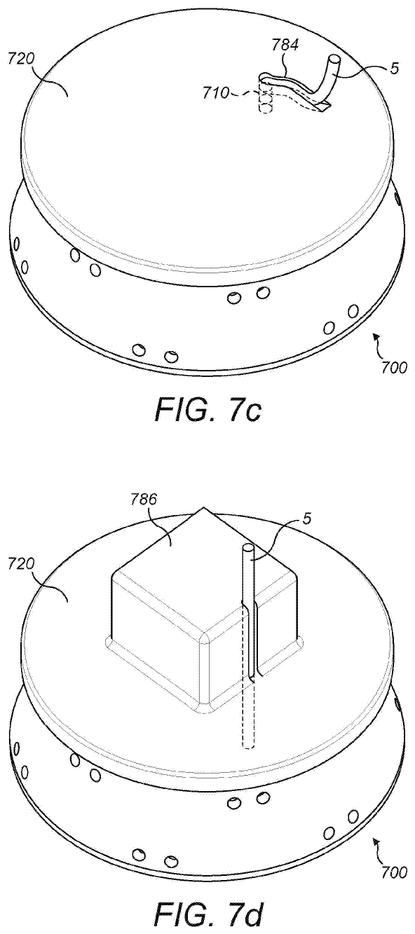

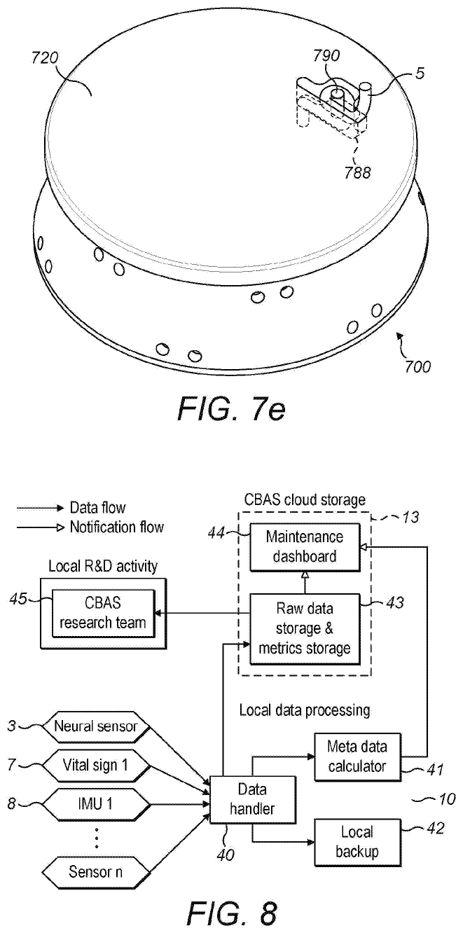

FIGS. 7a to 7e are top, side views of an interface device depicting additional preferably features. In these embodiments, various channel specifications are depicted to provided as retaining/supporting means for the one electrical connections 5 passing through the interface devices and will accordingly be described. However, it will be understood that modifications to and/or combinations of any of these embodiments is contemplated herein.

FIG. 7a illustrates interface device 700 comprising a channel 780 on the cap portion 720 extending from the port 710 configured to receive and/or retain one or more electrical connections 5 extending therefrom. The channel 780 may have a uniform depth or may have a slant, sloped or curved depth.

FIG. 7b illustrates an alternative embodiment of interface device 700 wherein the sides of channel 780 narrow at a point along the channel 780 such that the one electrical connections 5 extending from the port 710 are retained/secured in place.

FIG. 7c illustrates an alternative embodiment of interface device 700 wherein the channel 784 has a kink, wriggle or wave so as to secure or retain the one or more electrical connections 5 therein.

FIG. 7d illustrates an alternative embodiment of interface device 700 further comprising a retaining means 786 extending along side the port 710 to provide a further mechanical means for holding/supporting the one or more electrical connections 5. Although in this embodiment, the support or retaining means 786 comprises a box type structure with a groove or channel for encasing the one or more electrical connections 5, it will be appreciate that any other suitable shape is possible. For example, the support means 786 may simply be comprise a frame or support beam to secure the one or more electrical connections 5 along any number of sides; although preferably along at least two sides.

FIG. 7e illustrates an alternative embodiment of interface device 700 wherein the channel 788 is configured about a post 790 to enable the one or electrical connections 5 to wrap around the post for retention thereby.

Although in each of the aforementioned embodiments the channels are recessed into the cap portion of the interface device, it will be appreciated that this is not necessary and alternatively or additionally could be provided with mechanical means to hold the one or more wires, cables and/or tubing in place.

The interface device can be 3-D printed.

### Data Handling Architecture

FIG. 8 shows a schematic diagram of the architecture used to collect and store the data gathered by the system 1. The architecture comprises a software system that curates, processes and transmits the data to the central data storage system 13. Data acquired by the sensors implanted in, and located on, the subject animal 2, such as the sensors 3 and 7 to 9, and any other sensors, together with activity data from the active device 17 and environmental modification data from the environmental modification device 18 is sent through the wireless communication devices 11 and communication network 12 to the local computer 10. The video data from the video cameras 14 is also sent through the communication network 12 to the local computer 10.

The local computer 10 carries out data recording on the received data signals and acts as a data handler for all data relating to the subject animal 2. The local computer 10 comprises a data handler module 40 which carries out data recording on the received data, applies time stamps to the received data, and sends the data to the central data storage system 13 for storage together with the associated time data in the form of the applied time stamps. The local computer 10 also comprises a meta data calculation module 41 which calculates metadata relating to the received data and/or sent data, and sends a notification to the central data storage system 13 if the calculated metadata indicates any issues. The functional of modules 40 and 41 of the local computer 10 may be provided by software modules running on the local computer 10. The calculated metadata is also be sent to the central data storage system 13 for storage together with the data.

The local computer 10 may also send data regarding any instructions sent to the active device 17 and/or environmental modification device 18 by the local computer 10, together with associated time data in the form of time stamps, to the central data storage system 13 for storage. The local computer 10 may also send data regarding any acknowledgement or feedback information received from the active device 17 and/or environmental modification device 18 by the local computer 10 to the central data storage system 13 for storage together with associated time data in the form of time stamps. The local computer 10 may also send data regarding any active neural stimulus or pharmaceuticals administered to the subject animal 2 together with associated time data in the form of time stamps to the central data storage system 13 for storage.

The local computer 10 may also enable the manual input of data

The local computer 10 also maintains a temporary local backup 42 of the data recently sent to the central data storage system 13. This temporary local backup 42 may be used to avoid any permanent loss of data in the event that any data sent to the central data storage system 13 is, for some reason, lost or corrupted in transit and not properly received.

The central data storage system 13 comprises a data store 43 and a maintenance system 44. The data store 43 stores the data and metadata received from the local computer 10. The maintenance system 44 receives any notifications from the local computer 10 and takes any necessary remedial action, and/or issues alerts to a system operator as appropriate. Conveniently, the data store 43 or the entire central data storage system 13 may be a cloud based system. This may be desirable in order to allow the very large amount of obtained neural data and other data to be stored, and to facilitate remote access.

The data stored in the data store 43 may be accessed at any time when it is desired to carry out analysis or research on the data, for example, the stored data may be accessed by a research team computer 45. The research team computer 45 may carry out analysis or research on stored data retrieved from the data store, for example by applying machine learning elements to the retrieved data.

The data stored in the data store 43 may be accessed for automated or manual processing and/or analysis, and the resulting processed data may then itself be stored in the data store 43. This may allow synergy and improved efficiency by avoiding the need for the same processing or analysis to be carried out on different occasions, or by different entities, such as different research teams. The stored data may be processed and/or analysed using any suitable method.

In some examples, the stored data may be processed and/or analysed using a machine learning (ML) model, or models. Machine learning and machine learning models are known to the skilled person in the technical field of the present invention, and need not be described in detail herein.

Machine learning (ML) models may be used to process and analyse the stored data in order to determine information of interest. In some examples, an ML model may be used to calculate or determine a physiological parameter value or bodily variable of the subject animal. The determined physiological parameter value or bodily variable can then be stored and/or displayed.

The determined bodily variables can include bodily variables that are not directly measureable, or are not readily, or easily, measurable, so that displaying the determined bodily variable provides useful information about the state of the subject animal which is not otherwise available, or is not readily, or easily, otherwise available. Thus, displaying the determined bodily variable can provide useful information about the state of the subject animal which is not otherwise available, or can only otherwise be obtained with difficulty.

In some examples the at least one physiological parameter value or bodily variable is at least one of: heartrate of the subject animal; activity of the subject animal; temperature of the subject animal; blood glucose level of the subject animal; any vital sign of the subject animal; any physiological measurement of the whole of the subject animal, a body part of the subject animal or a sub-part of the subject animal; any data representative of a state of the whole of the subject animal, a body part of the subject animal, or a sub-part of the subject animal.

Further, an ML model can determine a health of the subject animal, for example by monitoring across one or more physiological parameters or recorded neural data channels, and provide warnings if something is wrong, or beginning to go wrong. This may enable health issues to be identified before they could be identified from observing the physiological parameters themselves, or enable health issues to be identified which cannot be identified from observing the physiological parameters themselves. This enables the state of the subject animal to be understood at a deeper level than can be understood by conventional observation, for example by a human observer reviewing video recordings of the subject animal or viewing the subject animal directly.

This capability to provide an understanding at a deeper level of the state of a subject animal may be particularly useful and relevant for monitoring a disease state in a subject animal.

For example, when the subject animal is coming round from anesthesia the ML model can determine, based on analysis of the neural data, when the anesthesia effects have cleared before this can be observed and appreciated by a human observer.

The data is stored in the data store 43 in a loose file structure with file names that identify the sensor channel, time of recording and epoch. Recordings are taken in short data chunks, for example a few minutes, allowing for ease of file handling. Each chunk may be subdivided into multiple sequential files depending on sensor data rate. Databases then store times of discrete experiment events, signal quality and other metrics over the lifetime of the study and allow querying based on finding periods of certain channels, signal quality or in relation to certain events. This provides a flexible way of sampling sections of the data based on the investigators chosen study. This enables neural data and parameter data to be recovered and referenced by subject animal or across multiple sensors, or based on time periods or stored event data. These database queries are also accessible by a front-end user interface for ease of use which allows data to be exported in multiple formats, with various time bases, or in certain sized chunks.

The above description refers to processing the stored data. It should be understood that in some examples the stored data may be processed immediately it has been stored. In some examples this may result in processing which appears to be in real time, or near real time, to human observers.

The illustrated example shows only a single local computer 10 associated with a single subject animal 2, for simplicity. In practice the system 1 may comprise a plurality of subject animals and a plurality of local computers. In some examples there may be a one to one association of local computers to subject animals. In other examples there may be multiple animals associated with each local computer, or multiple local computers associated with each subject animal. The illustrated example has a single local computer 10 and a single central data storage system 13. In some examples the system 1 may comprise a plurality of local computers 10 connected to a single central data storage system 13.

The illustrated example uses a single local computer to acquire data and to send instructions to active devices and/or environmental modifying devices. In other examples these functions may be carried out by separate local computers.

The data handling architecture utilizes hashing checksum comparison at each data transfer point for checking data integrity.

As a part of the data acquisition functionality the local computer 10 runs timing software that synchronizes and time stamps all data streams and partitions file sizes into similar sized chunks prior to upload to the central data storage system 13. As different sensor types sample at vastly different frequencies this is important for maintaining file size parity and improving uploader performance. Timing software also controls discrete events being scheduled such as stimulation patterns for testing nerve conduction health and remotely triggered sensor recalibration events.

In the illustrated examples the time stamps applied by the local computer 10 are used as the associated time data for the stored data. In alternative examples the data may be time stamped before it is received by the local computer 10. In some examples, some, or all, data acquired by the sensors, including the neural transducer and video cameras, can be time stamped by the sensors themselves before transmission. Similarly, in some examples some, or all, data and/or notifications from active devices and/or environmental modification devices may be time stamped by the devices themselves before transmission. In some examples the data and/or notifications may be time stamped by the data processing unit 4c of the external module. In some examples the data and/or notifications may be time stamped by the wireless receivers 11, or by elements of the communication network 12. In examples where tine stamping is carried out outside the local computer 10, tome clocks in the time stamping components, such as sensors, wireless receivers, video cameras, active devices and/or environmental modification devices, can be synchronised to the local computer 10, or to a remote time source, to ensure time synchrony across the different parts of the system.

The local computer 10 software uses an asynchronous upload script to upload data to the central data storage system 13. This may ensure that the system is flexible to bandwidth changes. The temporary local backup 42 is used may be used to buffer data if there are upload issues. Preferably, the local backup 42 has sufficient capacity to buffer up to several days of data.

Preferably, the data acquisition software collecting the data is run in multiple instances across the, or each, local computer 10, and each local computer 10 may be connected to all the sensors from one subject animal, all the sensors of one type, or any combination of sensors. Regardless of the chosen relationship between local computers and the data streams from the different sensors and subject animals, configuration files ensure that each sensor input is labelled according to type and subject animal so that after files from all local computers 10 have been united in the central data storage system 13 the files and data can be readily indexed by database systems managing the file distribution.

The system 1 may be linked to external communication networks or systems to enable the system 1 to send, or push, communications to specific destinations in response to the identification of predetermined specific events by the system 1. For example, when the system 1 is being used to conduct a research project or experiment the system may send messages, for example by email or SMS, to specific destinations, such as individuals, involved in the research when specific events are identified in the stored data.

The communications may be generated by the local computer 10, or by the central data storage system 13. The communications may be sent in response to events such as the sensed data indicating that one or more parameters of the subject animal 2 have passed a threshold or thresholds, or that specific actions have been carried out by the active devices and/or environmental modification devices. The communications may also be sent out in response to predetermined events of the system 1, such as a low stored power level in the external module, or the metadata indicating a failure of a sensor or other component.

Preferably, the data handling architecture is laid out according to a “dumb client, smart server” design basis that means that local computers are agnostic of the type of data they are collecting or which subjects they are collecting data from and data is united within the central data storage system 13. This enables the data handling architecture and the system to be modular and scalable across multiple subjects, sensor types and even across multiple geographic locations.

The data gathered by the system 1 may be subject to range of processing between the initial sensing from which the data is derived and the storing of the data in the data store 43. This processing can take place at any point in the system 1, or may be distributed across several points. For example, the processing may be shared between the local computer 10, the central data storage system 13, and/or the sensors themselves.

On uploading of data to the central data storage system 13 a flexible load balanced validation script in the central data storage system 13 checks the contents of each data file to check if valid data has been recorded. These validation checks look at various signal metrics including amplitude, frequency spectrum, power spectrum, etc. to check that each is within reasonable bounds individual to each sensor. The results of these validation checks may be used as an additional quality metric of each data channel at all points in time.

On the local computer 10 data files are post-processed to allow splitting of data files into appropriate segments of time and into individual data files for each sensor channel. For example splitting accelerometer and gyroscope data from an IMU sensor, or splitting data for different neural channels that correspond to different physical implant locations along a nerve from a neural sensor.

Local processing at the local computer 10 is used to change data save file format or remove/alter compression to allow ease of use in post experiment analysis and for doing other data validation checks.

Processing scripts can also be used in the central data storage system 13 to run simple data analysis that is then stored alongside raw data to give a data overview, making subsequent parsing of the stored datasets simpler, enabling picking segments of data when conducting R&D or similar tests. Analyses that could be used include averaging over seconds or minutes to provide a readable summary, or noting periods of high variability

Possible preferred locations in the system for different parts of the processing are identified above. However, the processing may be carried out at other locations in the system in some cases. The processing described above is by way of example only, and may not be carried out in some examples.

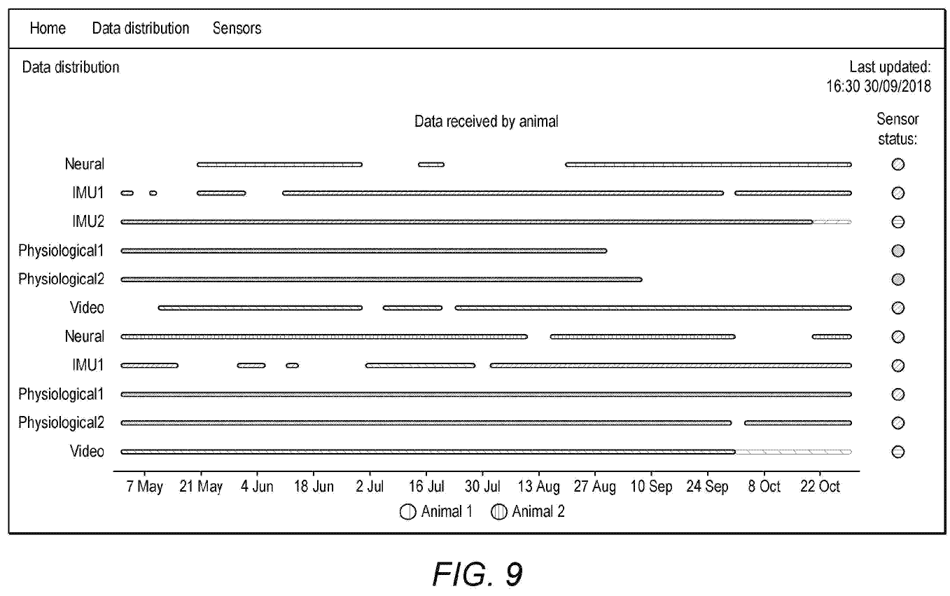

FIG. 9 shows an example of a status monitoring and data overview screen 800 which may be provided to users as a front end maintenance dashboard by the maintenance system 44. The front end maintenance dashboard provides notifications of problems and status updates when data is being gathered by the system 1, for example during an experiment. The front end maintenance dashboard also provides a view of the metadata relating to the obtained data in a concise format while the data is being gathered, or after data gathering has stopped, for example after the experiment is concluded. This may be useful in order to guide search efforts when picking data sections for R&D, or similar studies.

As shown in FIG. 9, the front end maintenance dashboard displays a timeline of activity for each sensor showing if data was collected and passed validation checks. The timeline is displayed for each sensor only at times for which data has been received and validated.

As shown in FIG. 9, The front end maintenance dashboard shows the upload status of data files awaiting uploading, for example in an upload queue. The front end maintenance dashboard also displays the operational status, for example battery level, of the sensors. In examples where a plurality of sensors are powered by the power and wireless communication module the battery level of the power and wireless communication module may be displayed.

The front end maintenance dashboard displays discrete events on the timeline together with the associated meta data. These discrete events can be sensor related events, events relating to active devices, or environmental modifications. Examples of events include a nerve being stimulated, a battery of a sensor changed, or a subject animal being fed.

Recorded events can also include events external to the system 1, such as administration of a medical intervention to the subject animal. The recording of such external events may be carried out manually, or by a special reporting system, not shown.

In FIG. 9, solid horizontal lines represent periods of continuous data capture from each sensor, hashed horizontal lines show when data is being captured by the local sensors but has been delayed in uploading. This representation allows easy identification of data drop issues during study or afterwards to find periods where all data streams were capturing for targeted R&D or similar data studies. In some examples the display can show: battery status of each device; show signal quality on each channel; allow scrolling in time to investigate at higher resolution; mark discrete events for each subject (e.g. feeding, medical checks, sensor calibration events).

The system may further comprise a user front end to enable users to view and analyze the data in the data store 43. The user front end may be remotely accessible by remote users, for example using a browser. The user front end may be used to enable users such as users of the research team computer 45 to access and analyze the stored data in the data store 45.

The user front end is arranged to enable the neural data and parameter data to be viewed together in a time synchronous manner, and may also enable this data to be viewed in a time synchronous manner with other data such as delivered neural stimulation, delivered treatment, or other events. Data regarding delivered neural stimulation, delivered treatment, or other events may be derived from the stored activity data provided by the active devices and/or environmental modification devices.

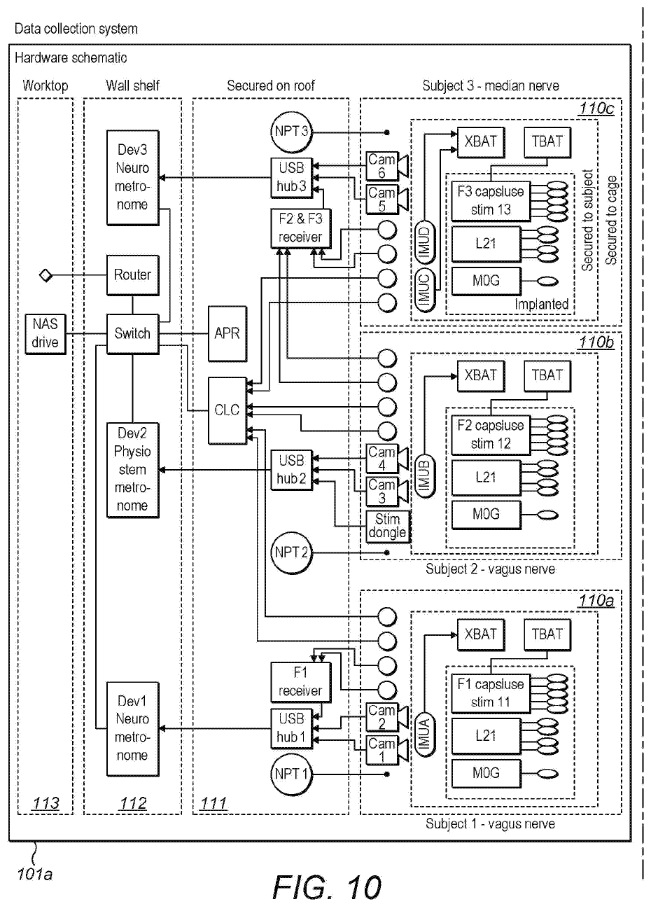

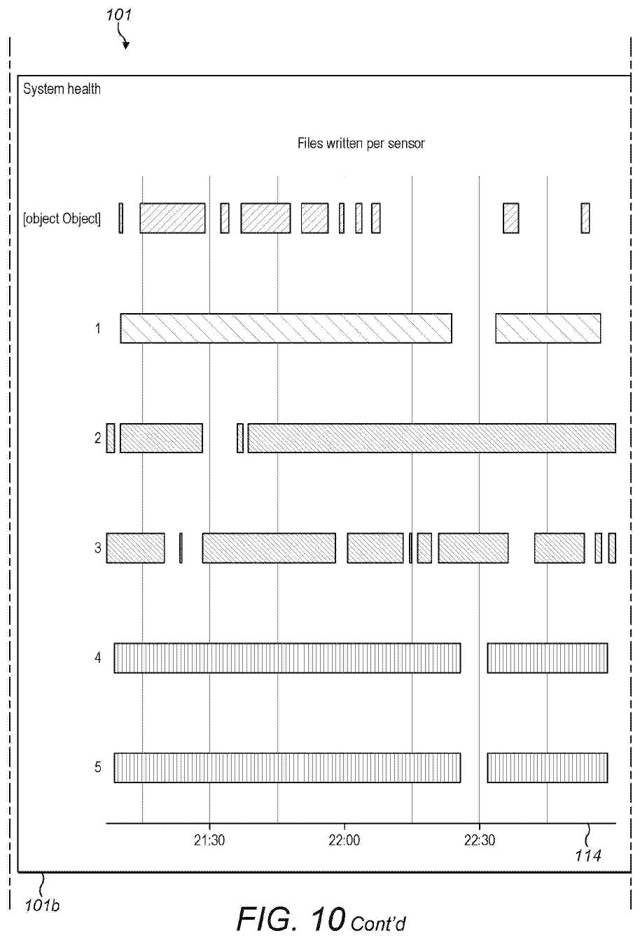

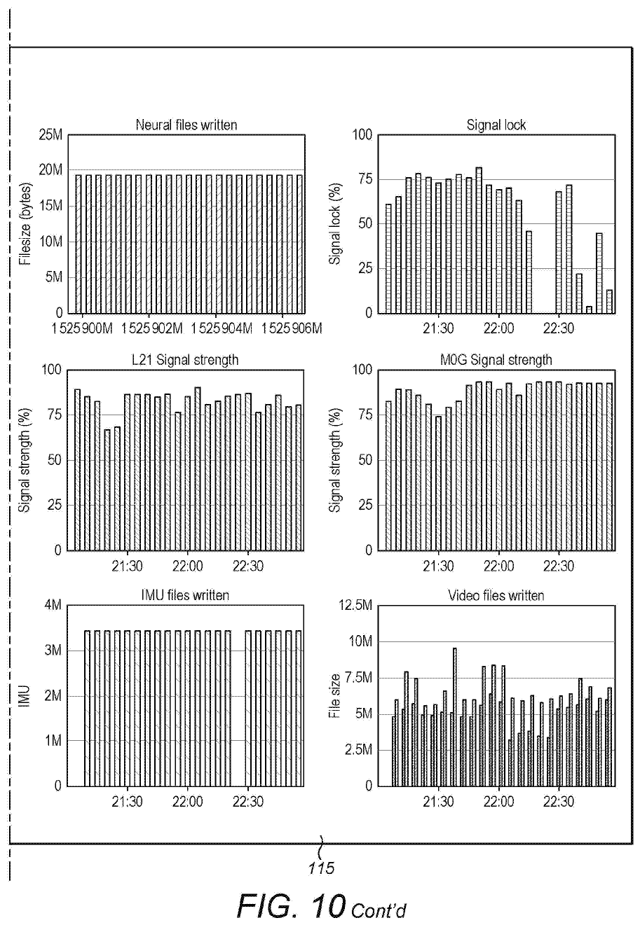

FIGS. 10 to 12 show examples of different first to third display screens 101 to 103 which may be displayed by the user front end, or other computers or devices accessing the data in the data store 43, in order to assist users in visualizing and analyzing the data. In some examples two, or all, of the first to third display screens 101 to 103 may be displayed simultaneously to a user or users.

FIG. 10 shows an exemplary first display screen 101 showing information relating to the data collection system 1 itself. The first display screen 101 comprises a first section 101a showing a schematic diagram of the current arrangement of the hardware elements of the data collection system 1 and a second section 101b diagrammatically showing the current performance of the different elements of the data collection system 1.

In the illustrated example the first section 101a of the first display screen 101 comprises regions 110a to 110c relating to different subject animals 2, with each of the regions 110a to 110c showing the data gathering devices associated with the specific subject animal, such as implanted sensors, external sensors, external module elements, and cameras, and schematically illustrating the statuses, and data communication links between, these data gathering devices and other parts of the system 1. In the illustrated example, the first section 101a further comprises regions 111, 112 and 113 relating to parts of the system 1 at different locations, and schematically illustrating the statuses, and data communication links between, these system elements.

In the illustrated example the second section 101b of the first display screen 101 comprises a first region 114 showing the data capture performance of different data gathering devices over time. Typically, the region 114 shows the performance of the same data gathering devices which are shown in the first section 101a, although this is not essential. The data capture performance of the different data gathering devices is shown in the region 114 in a similar manner to that described above with reference to FIG. 9, as a timeline of activity for each sensor showing if data was collected and passed validation checks. The timeline is displayed for each sensor only at times for which data has been received and validated. In the illustrated example, the second section 101b further comprises a region 115 showing the performance of all, or a selected part of, the data collection system 1. Typically, this is the selected part of the data collection system 1 shown in the first section 101a although this is not essential. The region 115 shows values over time of a number of performance metrics of the data gathering device and/or the data provided by the data gathering device. In the illustrated example the displayed performance metrics include the number of neural files written, signal lock percentage, transmitted and received signal strength of selected system components, number of inertial measurement unit (IMU) files written, and number of video files written. These displayed performance metrics are only examples, and other performance metrics may be used.

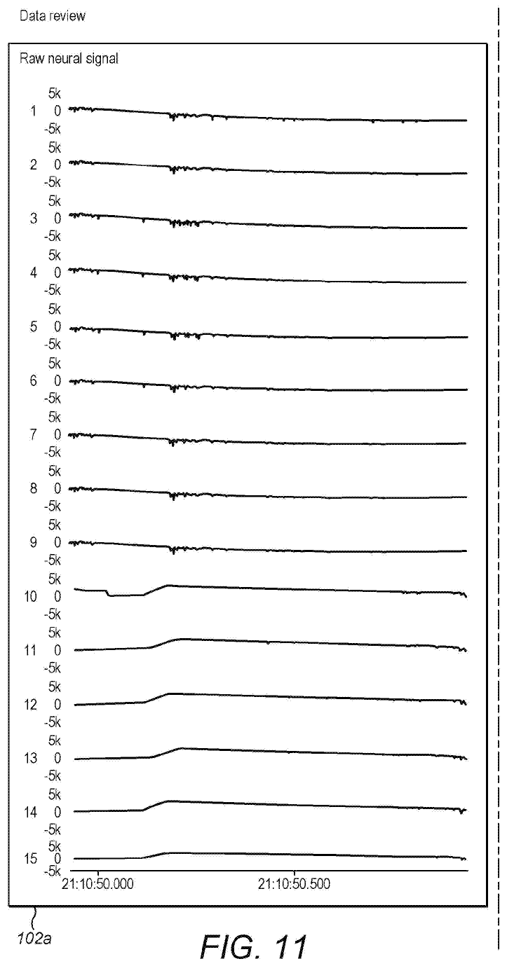

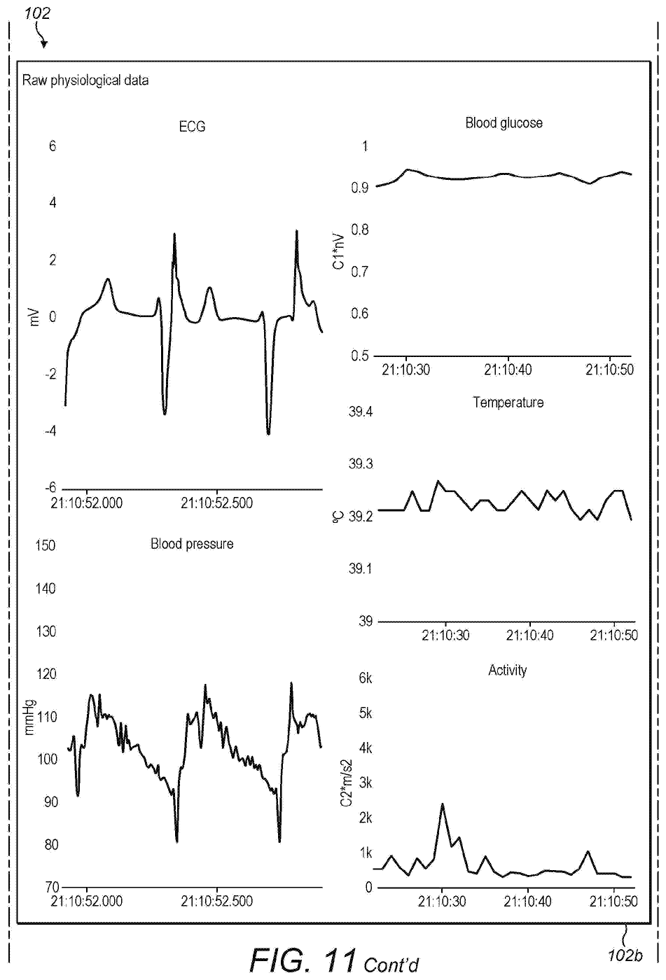

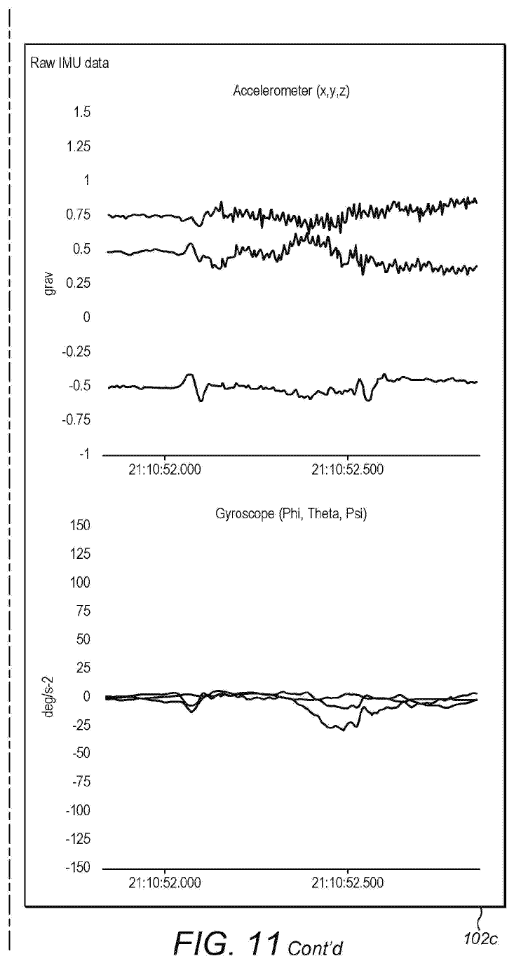

FIG. 11 shows an exemplary second display screen 102 showing raw data collected by the data collection system 1. The second display screen 102 comprises a first section 102a graphically showing raw neural signal values over time produced by different ones of the neural sensor module, of the data collection system 1. Typically, these are the neural sensor modules shown in the first section 101a, although this is not essential. The second display screen 102 further comprises a second section 102b graphically showing raw physiological data values over time produced by different sensor modules of the data collection system 1. Typically, these are the sensor modules shown in the first section 101a, although this is not essential. In the illustrated example the displayed physiological data values include ECG values, blood pressure values, blood glucose level values, body temperature values, and activity values .These displayed physiological data values are only examples, and other physiological data values may be used. The second display screen 102 further comprises a third section 102c graphically showing raw IMU data values over time produced by different motion sensor modules of the data collection system 1. Typically, these are motion sensor modules shown in the first section 101a, although this is not essential. In the illustrated example the displayed motion sensor data values include accelerometer values and gyroscope angle values. These displayed motion sensor values are only examples, and other motion sensor values may be used.

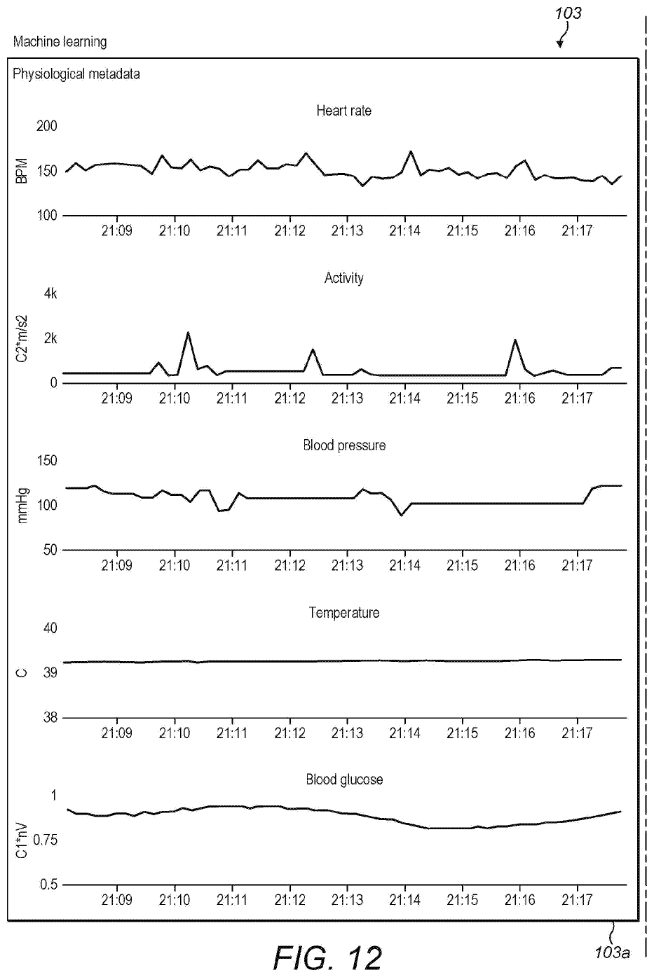

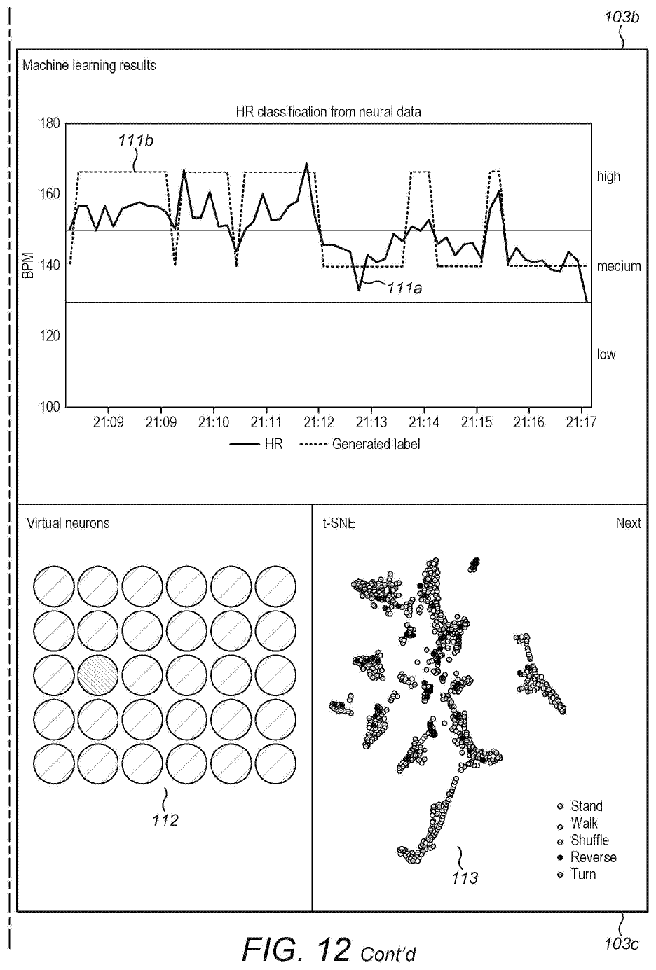

FIG. 12 shows an exemplary third display screen 103 showing information regarding operation of a machine learning model or models of the machine learning element or elements of the system 1. The third display screen 103 comprises a first section 103a showing values of physiological metadata, a second section 103b diagrammatically showing information regarding the output of the machine learning model or models calculating physiological metadata values, and a third section 103c showing intermediate states of the machine learning model or models. Preferably, the information displayed on the second and third display screens 102 and 103 is synchronous, or substantially synchronous, that is, it relates to data obtained at the same time or time range.

In the illustrated example the first section 103a of the third display screen 103 shows values over time of one or more physiological parameters. In the illustrated example the first section 103a these physiological parameters include heart rate, physical activity, blood pressure, body temperature, and blood glucose values.

The second section 103b of the third display screen 103 graphically illustrates the results of a machine learning model determining a physiological parameter of a subject animal based upon neural data obtained from the subject animal by graphically displaying over time a measured value 111a of the physiological parameter and a predicted or determined value 111b of the same physiological parameter as predicted or determined by the machine learning model based upon neural data. In the illustrated example the machine learning model takes in raw neural data from a subject animal and uses this to predict a heart rate classification of the subject animal. In the illustrated example the second section 103b shows the measured value of the heart rate over time and a corresponding classification of the heart rate as high, medium or low, as predicted based on neural data by a machine learning model. The illustrated example is an example of a supervised machine learning model with labels.

Displaying a comparison of the measured heart rate, or other physiological parameter, value to the value or classification predicted or determined from neural data by a machine learning model over time may provide a user with information regarding the quality of the data provided by the output of the machine learning model. This may enable a user to more readily identify anomalies or changes in the data provided by the output of the machine learning model and how these correspond to changes in the corresponding physiological parameters.

The third section 103c of the third display screen 103 shows in a first part 112 a graphical representation of a supervised machine learning model, and shows in a second part 113 a graphical representation of an intermediate state of an unsupervised machine learning model showing feature outputs. Comparing these graphical representations shown in the first part 112 and the second part 113 may assist a user in associating the unsupervised classes to the desired outputs. The graphical representation of an intermediate state of an unsupervised machine learning model may indicate to a user how the unsupervised classes are derived and what they correspond to, and may enable the user to identify how many meaningful dimensions are represented in the data. The second section 103b and the third section 103c relate to the same machine model.

In the illustrated example of the first part 112 of the third section 103c shows virtual neurons of a supervised machine learning model presenting a 1×30 vector of supervised classes. These can be back-related by a user to labelled conditions. Further, in the illustrated example the second part 113 of the third section 103c is a t-SNE plot. A t-SNE plot is a known plotting technique used to reduce dimensionality of data and to display multiple dimensional data on a two dimensional (2D) display. In the illustrated example the t-SNE plot shows data in the same class as dots of the same colour, and the grouping and splitting of the data identified as being in different classes in the plot can indicate to a user how separate the different classes are.

The data displayed to a user by the different parts of the second and third display screens 102 and 103 may be regarded as a hierarchy of data comprising raw data (for example the raw physiological data and neural signals) and the final abstracted data (for example the heart rate classification), and also displaying intermediate metrics and/or data (such as the t-SNE plot) relating to the operation of the machine learning model. The display of this data hierarchy relating to the same physiological parameters and derived from physiological and neural data captured simultaneously may enable a user to better understand relationships between the different levels of the data hierarchy, and in particular may enable a user to understand how different data corresponds to different bodily variables or states of interest.

The intermediate metrics and/or data may relate to one or more activation layers of the network used for machine learning, or aspects of the operation, state, output data, input data performance or configuration of the machine learning method in use at the time the data is processed.

In the illustrated example virtual neurons of a supervised machine learning model presenting a vector of supervised classes is displayed. In other examples, other types of representation may be displayed showing simplified representations of equivalence to neural populations, neural events, or neural or non-neural biomarkers, or combinations of neural and non-neural biomarkers.

In the illustrated example a t-SNE plot is calculated and displayed. In other examples an alternative reduced dimensionality visual representation of the state of the machine learning (ML) model while processing some or all of the data may be calculated and displayed. In some examples a principal component analysis (PCA) plot, an independent component analysis (ICA) plot, or an Isomap may be calculated and displayed. This list is not intended to be exhaustive.

A bodily variable comprises or represents an end effect or tissue state describing a state of some portion of the body. The bodily variable may itself be classified as a sensory, control or other variable based on the role or function of this information and the use of it by the body. Bodily variables are transmitted to or from the central nervous system (CNS) via neural activity in portions of the nervous system. One or more instances of neural activity at one or more neural locations can be said to be an encoding of one or more bodily variables, portions thereof and/or combinations thereof. For example, neural activity of one or more neurons of nerve(s) may be generated or modulated by part of the body to encode one or more bodily variables for reception by other parts of the body, which decode the neural activity to gain access to the bodily variable, portions thereof and/or combinations thereof. Both encoding and decoding of bodily variables can be performed by the CNS and/or bodily tissues therefore facilitating transmission of information around the body of a subject. Bodily variables can be afferent signals transmitted towards the CNS for provision of information regarding the state of bodily variables or efferent signals transmitted away from the CNS for modifying a bodily variable at an end effector organ or tissue.

Examples of bodily variables in the organ system, and often encoded in the autonomic nervous system (ANS), could include low level parameters such as, by way of example only but is not limited to, current blood glucose concentration, temperature of a portion, part or whole of the body of a subject, concentration of a protein or other key agent, current fullness state of the bladder or bowel, current heart rate or blood pressure, current breathing rate, current blood oxygenation, instructions regarding insulin/glucagon production, instructions regarding heart pacing, instructions regarding blood vessel dilation or constriction for changing blood pressure, instructions regarding changing breathing rate, instructions regarding modifying alveoli dilation to modify oxygen concentration, instructions regarding modifying gastric activity, instructions regarding modifying liver activity, instructions regarding opening/closing of sphincters for voiding/retaining of the bladder or bowel. Medium level bodily variables could include current activity of a whole organ or organ construct and high level bodily variables would include measurements of whole bodily functions or actions such as sweating, defecating, hard breathing, walking, exercising, running etc. In the ANS, each instance of a bodily variable may be associated with a modified organ function, modifying an organ function, or modifying a bodily function (e.g. one or more bodily variable(s) or the state of an organ or tissue).

In another example, in the SoNS, one or more bodily variable(s) generated by the CNS may be transmitted via the PNS as efferent neural activity that is associated with one or more instances of motion (e.g. each bodily variable may be associated with a different motion or movement of a limb, contraction/extension of a single muscle fibre/fibre group/whole muscle/group of muscles, instructions to modify speed/strength length of a muscle contraction, and the like etc.) The CNS may also receive an afferent neural activity encoding a bodily variable corresponding to sensory neural information (e.g. a sensory bodily variable), where in this case the sensory bodily variable represents an encoding of sensory information such as, by way of example only but is not limited to, temperature or pressure on a section or portion of skin, the state of a limb or other muscle group including, angle or position of a joint, position of a whole limb or section of the body, an abstract parameter of activity of the whole body or sub-part of the body, generated by one or more neuron(s) or one or more neuronal population(s) associated with the limb or other moving bodily part and the like. The CNS receives the afferent neural activity and then deciphers or decodes this neural activity to understand the sensory bodily variable(s) and responds accordingly.

Although several examples of bodily variables have been described, this is for simplicity and by way of example only, it is to be appreciated by the skilled person that the present disclosure is not so limited and that there are a plurality of bodily variables that may be generated by the body of a subject and which may be sent between parts of the body or around the body as neural activity. Although neural activity may encode one or more bodily variables, portions thereof and/or combinations thereof, it is to be appreciated by the skilled person that one or more bodily variables of a subject may be measurable, derivable, and/or calculated based on sensor data from sensors capable of detecting and/or making measurements associated with such bodily variables of the subject. It is also to be appreciated by the skilled person that a bodily variable is a direct measurement of any one parameter and could be represented as a generalised parameter of activity or function in an area. This would include bodily variables such as mental states which can not be easily related to low level function such as, depressed, having an epileptic fit, anxious, having a migraine.

Although the term bodily variable is described and used herein, this is by way of example only and the present disclosure is not so limited, it is to be appreciated by the skilled person that other equivalent terms may be used in place of the term bodily variable, or used interchangeably or even in conjunction with the term bodily variable, including, by way of example only but is not limited to, the following terms of: vital sign(s), which may be used by clinicians to describe parameters they use for patient monitoring, such as by way of example only but is not limited to, ECG, heart rate, pulse, blood pressure, body temperature, respiratory rate, pain, menstrual cycle, heart rate variation, pulse oximetry, blood glucose, gait speed, etc.; biomarker, which may be used by biologists to describe, by way of example only but is not limited to, protein levels, or measurable indicator of some biological state or condition etc., this term has also been adopted by the Deep Brain Stimulation & Spinal Cord Stimulation clinical fields; physiological variable/physiological data, which may often be used by scientists to describe things like ECG, heart rate, blood glucose, and/or blood pressure and the like, this term is also used by Data Sciences International who make implants for recording physiological variables such as ECG, heart-rate, blood pressure, blood glucose, etc.; or any other term describing a number, metric, variable or information describing some state of the whole body of a subject, any part and/or subpart of the body of the subject and the like.

Although examples of bodily variables are given herein, this is by way of example only and the description is not so limited, it is to be appreciated by the skilled person that the list of bodily variables is extremely large and could, for all intents and purposes, be infinite because a bodily variable may be, by way of example only but is not limited to, any number, parameter, metric, variable or information describing some state of the whole body of a subject, any portion, part and/or subpart of the body of the subject and that a bodily variable may be based on, or derived from, one or more combinations of one or more bodily variables or other bodily variables and the like. For example, bodily variables may be described at different levels of granularity in relation to a subject. There may be various different levels of granularity with multiple lower levels of granularity and multiple medium to higher levels of granularity in which bodily variables. Bodily variables at one or more lower levels of granularity may comprise or represent one or more bodily variables describing, by way of example only but not limited to, something about the body, part or subpart of a body of a subject at a neurological level, biomarker level, cellular level, and/or tissue level, modifications thereof, and/or combinations thereof and/or as herein described. Bodily variables at one or more higher levels of granularity may comprise or represent one or more bodily variables describing, by way of example only but not limited to, something about the vital signs of a subject; physiological meta data of a subject; sensor data representative of one or more bodily variables describing something about the body, parts of the body, or whole body of the subject; state, motion, or output of the body, part of subpart of the body of a subject and the like; modifications thereof, and/or combinations thereof and/or as herein described. Furthermore, one or more bodily variables described at one or more higher levels of granularity may be based on a combination of one or more bodily variables described at one or more lower levels of granularity. As can be understood from the above, some bodily variables may not be directly measurable, or may not be readily, or easily, measurable.

Displaying intermediate metrics and/or data regarding a machine learning model together with the time corresponding raw data or the final abstracted data provided to and produced by the machine learning model, and preferably both the time corresponding raw data and the final abstracted data, enables the relationships between these data at these different levels of abstraction to be more readily identified and understood. Further, such display enables body variables to be more readily matched to the different data, and in particular to the intermediate metrics and/or data.

The system described herein can provide long term monitoring of biological data, and in particular neural data, without impairing the natural movement and behavior of the subject animal. As explained above, the system can support a high bandwidth of data with a sustained data transmission rate, and can allow an effectively unlimited length of study, limited only by the health of the subject animal and the physical integrity of the sensors.

Potential uses of the system include the study of neural reactions to administered treatments, and research into different treatment regimes, either by the use of multiple subject animals, or by successive different treatments of the same subject animal. The system can also be used to conduct neural studies comparing neural data and data from other sensors, such as studies of diseases and controls.

The recording of sensor data from different sensors together with time stamps, or other time indications, in the data store simplifies comparing data and events at different times, so that causes and effects, and changes over time can be readily identified. Further, recording of sensor data and actions by active devices, such as the administration of pharmaceuticals, together with time stamps, or other time indications, in the data store allows controlled testing of pharmaceuticals and recording of the resulting neurological responses, and allows straightforward pairing of applied nerve stimulus and measured neural response.

In some examples the system may be used to administer pharmaceuticals based upon sensed neural data or other sensed physiological activities or parameters or behaviours, and then to record the results.

The system is able to conduct studies and research reliably and clearly combining neural, physiological and environmental data over time, and to collect large valid data sets for use in medical and biological research.

The types of analysis that can be conducted on neural data can include any one or more of: individual nerve and muscle activations; analysis of groups of muscles and nerves; dynamics of firing patterns of nerves or muscles including the timing of firing such as frequency, rate, interval, shape of firing signal and the distribution pattern across the population of neurons; and the overall changes in electrical potential of the tissue at one or more sites anywhere within the limb. It should be noted that combinations of any or all of the above may be used simultaneously to improve data quality and that which types of analysis are under use may change dynamically.

In the illustrated embodiments described above the through port connection of the skin interface device provides a wired connection between the system components inside the body of the subject animal and the external module, and specifically an electrical wired connection. In other examples the through port connection may be other types of connection.

In one example the through port connection may be an optical fiber connection. In such examples it may be necessary to provide electro-optical converters for the connected system components inside the body of the subject animal and the external module in order to convert between electrical and optical data signals. In some examples the implanted sensors may be optical in nature so that they do not require such converters.

In another example the through port connection may be a fluid or gas passage channel. In such examples it may be necessary to provide suitable converters for the connected system components inside the body of the subject animal and the external module in order to convert between electrical and fluidic data signals. In examples where the through port connection is a fluid or gas passage channel this may be used to provide pneumatic, hydraulic or fluidic power to system components inside the body of the subject animal from the power supply unit 4b of the external module 4. Alternatively, in examples where the through port connection is a fluid or gas passage channel this may be used to supply fuel to a fuel cell of a system component inside the body of the subject animal from the power supply unit 4b of the external module 4.

In some examples multiple different through port connections may be used. For example, a wired connection may be used to provide electrical power to system components inside the body of the subject animal from the power supply unit 4b of the external module 4 in combination with an optical fiber connection to carry data between the system components inside the body of the subject animal and the external module 4. In another example a fluid or gas passage channel may be used to provide pneumatic, hydraulic or fluidic power to system components inside the body of the subject animal from the power supply unit 4b of the external module 4 in combination with an optical fiber connection or electrical connection to carry data between the system components inside the body of the subject animal and the external module 4. Other combinations are also possible.

The central data storage system is described as central to indicate that it may be used with a number of subject animals and/or local computers. The term central does not imply anything regarding the physical location of the central data storage system.

In the illustrated examples the system comprises at least one neural sensor module, and the system gathers neural data, possibly together with other data. In other examples the system 1 may not comprise any neural sensor module and is used to collect data other than neural data.

In the illustrated examples the active devices and/or environmental modifying devices are controlled by instructions from the local computer. In other examples one, some or all of the active devices and/or environmental modifying devices may be controlled by instructions from other parts of the system or instructions which they have generated themselves.

In the illustrated examples the system 1 comprises three wireless communication devices 11a to 11c connected to the local computer 10 through a communications network. In other examples a different number of wireless communication devices may be used. Any wired or wireless communications systems or protocols may be used to connect the wireless communication devices 11a to 11c to the local computer 10. In some examples a single wireless communication device integrated into the local computer 10 may be used.

In the illustrated examples the mobile part 1a of the system 1 comprises an external module 4 secured to the exterior of the body of the subject animal 2 and comprising a wireless communication unit and a power supply unit. In other examples the power supply and wireless communications functions could be provided by separate modules. In other examples the wireless communications functionality could be provided by another part of the mobile part 1a of the system 1 and the power and wireless communication module could be replaced with a power module which just provides electrical power to other components of the mobile part 1a. In some examples the wireless communications functionality could be provided by the data collection capsule 3b.

In the illustrated examples the or each further sensor module which is not connected to the external module communicate wirelessly directly with the wireless communication devices of the static part of the system. In alternative examples the or each further sensor module could communicate wirelessly indirectly with the static part of the system by communicating with the external module, which could in turn communicate with the static part of the system. This may allow the wireless transmission power and power consumption of the further sensor module to be reduced. In some examples the further sensor modules may include some further sensor modules which communicate wirelessly directly with the wireless communication devices of the static part of the system and some which communicate wirelessly indirectly with the static part of the system through the external module.

In the illustrated examples batteries are used. In other examples different types of power storage unit may be used. In some examples the power storage unit may be a fuel cell.

In the illustrated examples the communications network and/or communications links between the local computer and the central data storage system may comprise gigabit switches.

In the illustrated examples the video cameras are arranged to view the subject animals from different angles. This may be preferred in order to ensure that all relevant activity of the subject animal is visible to at least one video camera. However, in some examples the video camera or cameras may not be arranged in this way.

In the illustrated examples the housing is formed of transparent plastics. In other examples the housing may be formed wholly, or in part, of other materials.

In the illustrated examples the mobile part of the system comprises sensors and active devices. In some examples combined devices able to operate as both sensors and active devices may be used. In particular, in some examples one, some, or all of the neural sensor modules may also be able to carry out neural stimulation.

In some examples the system may include additional sensors to sense parameters of the ambient environment and to provide data regarding these parameters to the local computer and/or the central data store. These additional sensors may be comprised in the mobile part or the static part of the system, as appropriate.

In the illustrated examples the active devices comprise their own power supplies. In other examples one, some, or all active devices may be supplied with power from the external module 4 using electrical connections 5.

In the illustrated examples the system 1 comprises a housing. This is not essential. In some examples the housing may be omitted. The housing may not be required if the subject animal is in an environment which limits movement of the subject animal to a defined area.

In the illustrated examples the electrical connections have at least three parts, with separate internal, external, and bridging sections. In other examples the electrical connections may have a different number of parts. In some examples the electrical connections may be continuous and not separated into parts.

In the illustrated examples the transcutaneous device is engaged with soft tissue. In some alternative examples the transcutaneous device may additionally comprise an osseointegrated portion allowing the transcutaneous device to be secured to a bone of the subject animal. In some examples this osseointegrated portion may take the form of a bone stem or extra-cortical plate(s).

It will be appreciated that the radius and/or the surface area of the interface device, and in particular the cap portion, can be any dimension.

In the illustrated embodiment the modules of the system are defined in software. In other examples the modules may be defined wholly or in part in hardware, for example by dedicated electronic circuits.

In the illustrated examples the processing is carried out on stored data. In some examples the stored data may be processed immediately it has been stored, and in some examples this may result in processing which appears to be in real time, or near real time, to human observers. Further, in some examples the data may be processed in parallel to being stored, so that the processed data is the same as the data stored in the data store, but has not actually been obtained from the data store.

In the described embodiments of the invention the system elements may be implemented as any form of a computing and/or electronic device.

Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

The computer executable instructions may be provided using any computer-readable media that is accessible by computing based device. Computer-readable media may include, for example, computer storage media such as a memory and communications media.

Computer storage media, such as a memory, includes volatile and non-volatile, removable and non-removable media implemented in any method or technology for storage of information such as computer readable instructions, data structures, program modules or other data. Computer storage media includes, but is not limited to, RAM, ROM, EPROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device. In contrast, communication media may embody computer readable instructions, data structures, program modules, or other data in a modulated data signal, such as a carrier wave, or other transport mechanism. As defined herein, computer storage media does not include communication media.

The system 1 may be distributed or located remotely and accessed via a network or other communication link (e.g. using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of preferred embodiments is given by way of example only and that various modifications may be made by those skilled in the art. Although various embodiments have been described above with a certain degree of particularity, or with reference to one or more individual embodiments, those skilled in the art could make numerous alterations to the disclosed embodiments without departing from the spirit or scope of this invention.

1. A system for obtaining biological data from a subject animal, the system comprising:
at least one neural transducer embedded internally within the subject animal and arranged for interaction with nerves of the subject animal;
at least one sensor arranged for sensing data of a parameter of the subject animal; and
an external module mounted externally on the subject animal and connected through a port to at least one of the at least one neural transducer or the at least one sensor;
wherein the through port connection comprises a sealed and ported through skin interface device.

2. The system of claim 1, wherein the through port connection is a wired connection.

3. The system of claim 1, wherein the through port connection is a fluid or gas passage channel.

4. The system of claim 1, wherein the through port connection is an optical fiber.

5. The system of claim 1, wherein the neural transducer is a neural sensor arranged for sensing neural data from nerves of the subject animal.

6. The system of claim 5, the system further comprising:
a wireless communication unit for wirelessly sending the sensed neural data to a data store;
means for sending the sensed parameter data to the data store; and a power supply;
wherein the external module comprises at least one of the wireless communication unit and the power supply.

7. The system of claim 6, wherein the data store is arranged to store the sensed neural data together with associated time data, and is arranged to store the sensed parameter data together with associated time data.

8. The system of claim 6, wherein the external module comprises the power supply and the wireless communication unit.

9. The system of claim 6, wherein the external module comprises at least one of: a data processor arranged for processing the neural data; a data storage arranged for storing the neural data.

10. The system of claim 6, wherein the system further comprises at least one wireless communication device for wirelessly receiving the sensed neural data from the wireless communication unit.

11. The system of claim 10, wherein the system further comprises at least one local computer arranged to record the sensed neural data received by the at least one wireless communication device and the sensed parameter data, and to send the sensed neural data and the sensed parameter data to the data store together with associated time data.

12. The system of claim 11, wherein the at least one local computer is arranged to calculate metadata based on the sensed neural data and associated time data, and the sensed parameter data and associated time data, and to send the metadata to the data store.

13. The system of claim 6, wherein the system further comprises the data store.

14. The system of claim 1, wherein the at least one sensor comprises at least one sensor embedded internally within the subject animal.

15. The system of claim 14, wherein the at least one sensor embedded internally within the subject animal comprises at least one of: a glucose sensor; a heart rate sensor, an ElectroCardioGram (ECG) sensor, a blood pressure sensor, a vascular pressure sensor, an airway pressure sensor; an intrapleural pressure sensor; a gastric activity sensor; a gastric PH sensor, an Electro Muscular Graph (EMG) sensor.

16. The system of claim 1, wherein the at least one sensor comprises at least one sensor mounted externally on the subject animal.

17. The system of claim 16, wherein the at least one sensor mounted externally on the subject animal comprises a motion sensor.

18. The system of claim 1, wherein the external module comprises a data processor arranged for generating the time data associated with the sensed neural data.

19. The system of claim 1, wherein the system comprises at least one active device arranged for operating to produce a change in the subject animal.

20. The system of claim 19, wherein the at least one active device comprises the neural transducer.

21-173. (canceled)
